(19) United States
(12) Patent Application Publication
(10) Pub. No.: US 2013/0131156 A1
(43) Pub. Date: May 23, 2013

(54) Cancer Treatment with Wortmannin Analogs

(71) Applicants: Scott Peterson, Woodinville, WA (US); Diana F. Hausman, Seattle, WA (US); Robert Kirkman, Yarrow Point, WA (US)

(72) Inventors: Scott Peterson, Woodinville, WA (US); Diana F. Hausman, Seattle, WA (US); Robert Kirkman, Yarrow Point, WA (US)

(73) Assignee: ONCOTHYREON INC., Seattle, WA (US)

(21) Appl. No.: 13/701,270

(22) PCT Filed: Jun. 3, 2011
(86) PCT No.: PCT/US11/39166

§ 371 Date: Feb. 6, 2013

Related U.S. Application Data
(60) Provisional application No. 61/351,559, filed on Jun. 4, 2010.
(60) Provisional application No. 61/416,037, filed on Nov. 22, 2010.
(60) Provisional application No. 61/425,689, filed on Dec. 21, 2010.
(60) Provisional application No. 61/425,690, filed on Dec. 21, 2010.

(51) Int. Cl. C07J 73/00

(52) U.S. Cl. CPC C07J 73/003
USPC 514/453

(57) ABSTRACT

Provided herein are certain therapeutically effective dosing regimens for treatment of cancers with wortmannin analogs.

### CROSS-REFERENCE

This application claims the benefit of U.S. Provisional Application No. 61/351,559, filed Jun. 4, 2010; U.S. Provisional Application No. 61/416,037, filed Nov. 22, 2010; U.S. Provisional Application No. 61/425,689, filed Dec. 21, 2010; and U.S. Provisional Application No. 61/425,690, filed Dec. 21, 2010, each of which is incorporated herein by reference in its entirety.

### BACKGROUND OF THE INVENTION

Phosphatidylinositol-3-kinase (PI-3K) signaling is activated in a broad spectrum of human cancers via multiple mechanisms, including the increased expression or activity of cell surface receptors that activate PI-3K, increased expression of the PI-3K catalytic subunit, as well as mutations that activate the catalytic subunit or suppress the capacity of the regulatory subunit to regulate catalytic subunit activity. In addition, loss of PTEN via mutation, deletion, or epigenetic suppression serves to drive the pathway downstream of PI-3K. In addition to the genetic and histological evidence for PI-3K pathway activation in human cancer samples, PI-3K activation has been shown to be oncogenic in mouse cancer models. Taken together, it is contemplated that PI-3K pathway activation contributes to human disease pathology including glioblastoma multiforme and prostate cancer.

### Glioblastoma Multiforme

Glioblastoma Multiforme (GBM) is the most common malignant tumor of the central nervous system, comprising approximately 50% of all malignant brain tumors. The incidence of GBM increases with age, with a median age at diagnosis of 64. Males have a higher incidence rate than females. Exposure to ionizing radiation and rare genetic syndromes are the only reported risk factors, yet the incidence rate continues to increase over time with an average annual percent increase of 2.6%.

GBM is known to be resistant to treatment, and despite the use of multiple therapeutic modalities patient prognosis remains poor. Standard treatment following maximal surgical resection generally includes daily temozolomide (TMZ) chemotherapy in combination with radiotherapy for six weeks, followed by six cycles of TMZ given for the first five days of every 28 day cycle.

Those diagnosed with GBM suffer from significant morbidity. Tumor location frequently results in disability from motor, speech, visual or cognitive impairments. These patients are also at increased risk of seizure and venous thromboembolism. Local therapies, such as surgery and radiotherapy may contribute to these disabilities. These issues create considerable burden for social supports and increase caregiver stress. As a result, quality of life diminishes significantly and can remain poor for the duration of the patient's life.

### Prostate Cancer

Prostate cancer is a leading cause of male cancer-related deaths worldwide. Apart from lung cancer, prostate cancer is the most common cancer in men, and the second leading cause of death among men in the United States. Androgens play an important role in the development, growth, and progression of prostate cancer, with the two most important androgens in this regard being testosterone, 90-95% of which is synthesized in the testes and the remainder (5-10%) is synthesized by the adrenal glands, and dihydrotestosterone (DHT), the primary androgen in prostatic tissues.

In many prostate cancer therapies, agents that block the action (anti-androgens) of endogenous hormones (e.g., testosterone) are highly effective and routinely used for the treatment (androgen ablation, deprivation or withdrawal therapy). While initially effective at suppressing tumor growth, these androgen ablation therapies eventually fail in many patients, leading to “castration resistant” or “hormone refractory” prostate cancer (“CRPC” or “HRPC”). Most, but not all, prostate cancer cells initially respond to androgen withdrawal therapy. However, with time, new populations of prostate cancer cells emerge that have responded to the selective pressure created by androgen ablation therapy and are refractory to it. Not only is the primary cancer refractory to available therapies, but cancer cells may also break away from the primary tumor and travel in the bloodstream, spreading the disease to distant sites.

The current standard of care for castration resistant prostate cancer is palliative in its intent, and includes analgesia, radiation, bisphosphonates, and chemotherapy such as mitoxantrone, cabazitaxel, docetaxel, abiraterone or sipuleucel-T with a number of these drugs being associated with an overall survival benefit. With the early commencement of androgen deprivation therapy and frequent use of PSA for monitoring disease progression, an increasing population of patients with castration-resistant disease is now more commonly identified by a rising PSA rather than by new disease or symptoms.

### SUMMARY OF THE INVENTION

Provided herein are methods for treating cancer using PI-3 kinase inhibitors. In one aspect, certain dosing regimens for treatment of cancers with PI-3 kinase inhibitors are described herein. Also described herein are biomarkers indicative of therapeutic efficacy of PI-3 kinase inhibitors for treatment of cancers. In certain embodiments, a PI-3 kinase inhibitor suitable for treatment regimens described herein is a wortmannin analog. In certain embodiments, a PI-3 kinase inhibitor suitable for treatment regimens described herein is an irreversible PI-3 kinase inhibitor.

Provided herein, in some embodiments, are methods for treatment of cancer comprising administration of PX-866 to a human in need thereof:

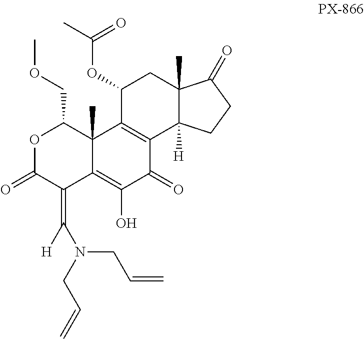 [SMILES: C=CCN(/C=C1/C(=O)O[C@H](COC)[C@@]2(C)C1=C(O)C(=O)C1=C2[C@H](OC(C)=O)C[C@]2(C)C(=O)CC[C@@H]12)CC=C]

at a dose and frequency of administration sufficient to result in a plasma concentration of a 17-hydroxy metabolite between about 500 pg/mL and about 2500 pg/mL (peak) within about 1-3 hours of administration of PX-866; wherein the 17-hydroxy metabolite has the structure:

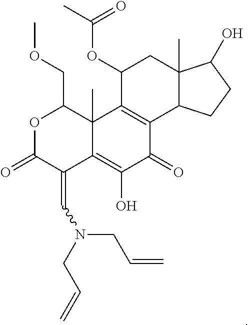 [SMILES: C=CCN(C=C1C(=O)OC(COC)C2(C)C1=C(O)C(=O)C1=C2C(OC(C)=O)CC2(C)C(O)CCC12)CC=C]

In some embodiments, PX-866 is administered to the human in an amount of from about 0.1 mg to about 20 mg per day. In some embodiments, PX-866 is administered to the human in an amount of from about 0.5 to about 16 mg per day.

In some embodiments, PX-866 is administered as a continuous dose. In other embodiments, PX-866 is administered as an intermittent dose. It further embodiments, PX-866 is administered as a combination of a continuous and intermittent dose.

In some embodiments, a continuous dose is between about 10% and about 85% of the Maximum Tolerated Dose (MTD) of the intermittent dose.

In some embodiments, administration of PX-866 provides a plasma Cₘₐₓ of the 17-hydroxy metabolite of between about 750 pg/mL and about 1750 pg/mL.

In some embodiments, administration of PX-866 provides an AUC of between about 2000 hr*pg/mL and about 8000 hr*pg/mL for the 17-hydroxy metabolite.

In some embodiments, the cancer is selected from anaplastic thyroid tumor, sacrcoma of the skin, melanoma, adenocystic tumor, hepatoid tumor, non-small cell lung cancer, chondrosarcoma, pancreatic islet cell tumor, esophageal cancer, prostate cancer, ovarian cancer, squamous cell carcinoma of the head and neck, colorectal carcinoma, glioblastoma, cervical carcinoma, endometrial carcinoma, gastric carcinoma, and breast carcinoma.

In some specific embodiments, the cancer is selected from anaplastic thyroid tumor, sacrcoma of the skin, melanoma, adenocystic tumor, hepatoid tumor, non-small cell lung cancer, chondrosarcoma, pancreatic islet cell tumor, esophageal cancer, prostate cancer, and ovarian cancer. In some specific embodiments, the cancer is glioblastoma. In other specific embodiments, the cancer is prostate cancer wherein the prostate cancer is castration resistant.

In some embodiments, continuous dose administration of PX-866 provides disease stabilization.

In some embodiments, PX-866 is administered as a continuous dose of between about 2 mg to about 12 mg per day. In some embodiments, PX-866 is administered as a continuous dose of between about 2 mg to about 10 mg per day. In some embodiments, PX-866 is administered as a continuous dose of between about 2 mg to about 8 mg per day.

In some embodiments, PX-866 is administered as an oral dose in fasting state. In some embodiments, PX-866 is administered as an oral dose in fed state.

In some embodiments, PX-866 is administered at a dose sufficient to avoid proteinuria and/or elevation in ALT/AST.

In some embodiments, PX-866 is administered in combination with corticosteroids, gamma-interferon, cyclophosphamide, azathioprine, methotrexate, penicillamine, cyclosporine, colchicine, capecitabine, mycophenolate mofetil, perfenidone, gefitinib, erlotinib, rapamycin, temsirolimus, deforolimus, everolimus, BEZ235, docetaxel, cetuximab, abiraterone, carboplatin, paclitaxel, cabazitaxel, gemcitabine, doxorubicin, daunorubicin, epirubicin, idarubicin, bevacizumab or radiation.

Also provided herein are methods of treatment of cancer comprising administration of a continuous dose of a PI-3 kinase inhibitor to an individual in need thereof. In some embodiments, the PI-3 kinase inhibitor is an irreversible PI-3 kinase inhibitor. In some embodiments, the PI-3 kinase inhibitor is PX-866, and/or a metabolite thereof. In some embodiments, the individual has undergone treatment with other cancer therapies (e.g., treatment with anthracyclines, paclitaxel/cisplatin or any other treatment) and has subsequent disease progression.

Also provided herein are methods for treating glioblastoma in a subject with a wortmannin analog. Provided herein also are methods for reducing glioblastoma tumor size in a subject with glioblastoma with a wortmannin analog. Also provided herein are methods of improving or maintaining the quality of life in a subject with glioblastoma with a wortmannin analog.

In one aspect, provided herein are methods for treating human subjects with a glioblastoma comprising administering to the subject a compound selected from

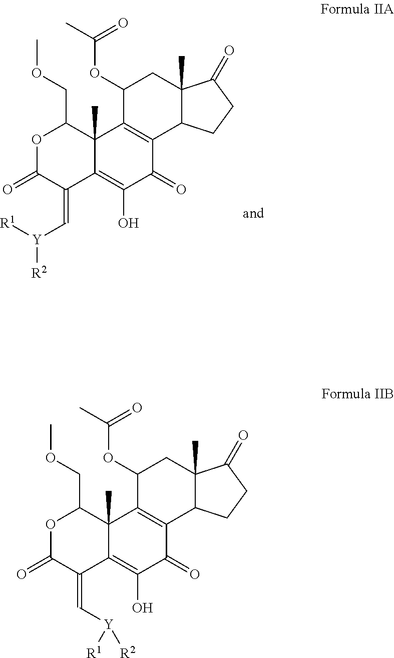

wherein Y is a heteroatom selected from nitrogen and sulfur and R¹ and R² are independently selected from an unsaturated alkyl, cyclic alkyl, or R¹ and R² together with Y form a heterocycle.

In some instances the glioblastoma is recurrent. In other instances, the glioblastoma is metastatic. In further instances, the glioblastoma is unresectable.

In some embodiments of the methods provided herein, administering of the compound is by injection, transdermal, nasal, pulmonary, vaginal, rectal, buccal, ocular, otic, local, topical, or oral delivery. In certain instances, injection is intramuscular, intravenous, subcutaneous, intranodal, intratumoral, intracisternal, intraperitoneal, or intradermal.

In certain embodiments, the compound is administered orally. In certain embodiments, the compound is administered in a capsule form. In certain embodiments, the compound administered is about 0.1 to about 12 mg. In certain instances, the compound is administered daily. In some instances, the compound is administered to the subject in a fasted state. In other instances, the compound is administered to the subject in a fed state.

In some embodiments, the administration is over a period of time selected from the group consisting of at least about 3 weeks, at least about 6 weeks, at least about 8 weeks, at least about 12 weeks, at least about 16 weeks, at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 68 weeks, at least about 72 weeks, at least about 90 weeks, at least about 100 weeks, at least about 110 weeks, and at least about 120 weeks.

In further embodiments of the methods provided herein, the compound is provided in a kit. In yet further embodiments, the methods provided herein further comprise an additional anti-cancer therapy. In yet further embodiments, the methods provided herein further comprise temozolomide. In yet further embodiments, the methods provided herein further comprise a corticosteroid. In yet further embodiments, the methods provided herein further comprise an anti-emetic, anti-diarrheal or both.

In some embodiments of the methods provided herein, the subject is preselected for having completed first-line anti-cancer therapy. In certain instances, the first-line anti-cancer therapy is surgery, radiation and/or chemotherapy.

In some embodiments of the methods provided herein, the subject is preselected for not having prior anti-cancer therapy with a PI-3 kinase inhibitor. In other embodiments, the subject is preselected for not having other active malignancies. In yet other embodiments, the subject is preselected for not having uncontrolled diabetes mellitus. In further embodiments, the subject is preselected for not being positive for human immunodeficiency virus (HIV).

In other embodiments, subject is preselected for sensitivity to administration of the compound. In certain instances, preselection is by assessment of genetic mutations in PI-3 kinase, PTEN, EGFRvIII and/or K-ras genes.

In other embodiments of the methods provided herein, the methods further comprise evaluating the treated subject, wherein the evaluation comprises determining at least one of: (a) glioblastoma size, (b) glioblastoma location, (c) nodal stage, (d) growth rate of the glioblastoma, (e) survival rate of the subject, (f) changes in the subject's glioblastoma symptoms, (g) changes in the subject's biomarkers, or (h) changes in the subject's quality of life.

In some embodiments of the methods provided herein, the compound, suitable for treatment of glioblastoma, is

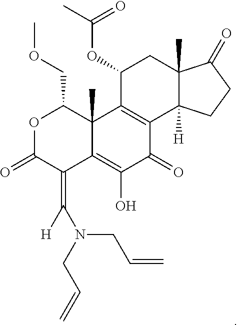 [SMILES: C=CCN(/C=C1/C(=O)O[C@H](COC)[C@@]2(C)C1=C(O)C(=O)C1=C2[C@H](OC(C)=O)C[C@]2(C)C(=O)CC[C@@H]12)CC=C]

In other embodiments of the methods provided herein, the compound, suitable for treatment of glioblastoma, is

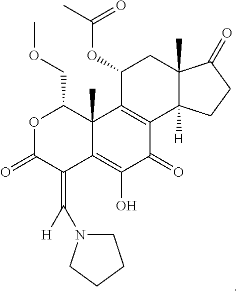 [SMILES: COC[C@H]1OC(=O)/C(=C/N2CCCC2)C2=C(O)C(=O)C3=C([C@H](OC(C)=O)C[C@]4(C)C(=O)CC[C@@H]34)[C@]21C]

In another aspect, provided herein are methods for reducing glioblastoma tumor size in a human subject diagnosed with a glioblastoma comprising administering to the subject a compound selected from

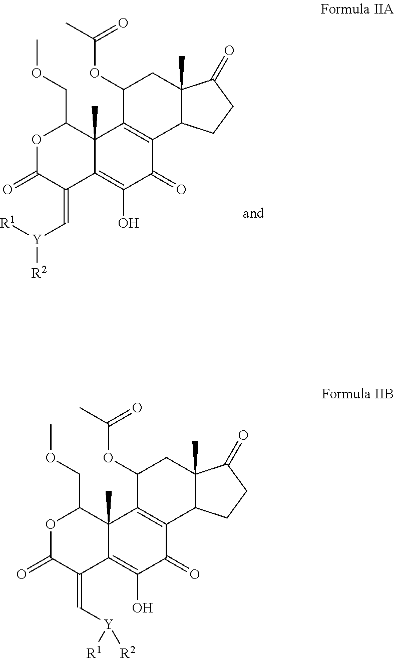

wherein Y is a heteroatom selected from nitrogen and sulfur and R¹ and R² are independently selected from an unsaturated alkyl, cyclic alkyl, or R¹ and R² together with Y form a heterocycle.

In yet another aspect, provided herein are methods for improving or maintaining the quality of life of a human subject diagnosed with a glioblastoma comprising administering to the subject a compound selected from

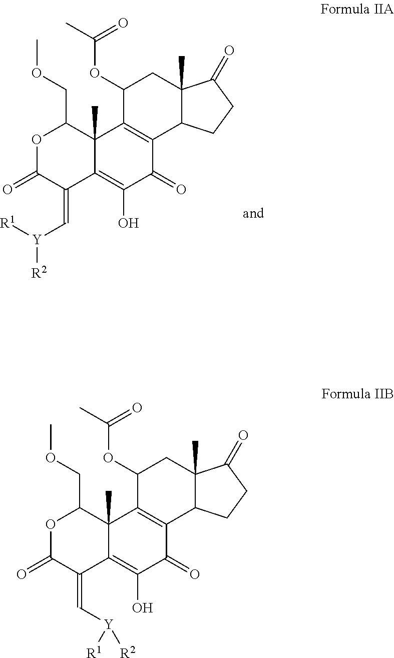

wherein Y is a heteroatom selected from nitrogen and sulfur and R¹ and R² are independently selected from an unsaturated alkyl, cyclic alkyl, or R¹ and R² together with Y form a heterocycle.

Also provided herein are methods for treating castration resistant prostate cancer in a subject with a wortmannin analog. Provided herein also are methods for reducing castration resistant prostate cancer tumor size in a subject with castration resistant prostate cancer with a wortmannin analog. Also provided herein are methods of improving or maintaining the quality of life in a subject with castration resistant prostate cancer with a wortmannin analog.

In one aspect, provided herein are methods for treating human subjects with a castration resistant prostate cancer comprising administering to the subject a compound selected from

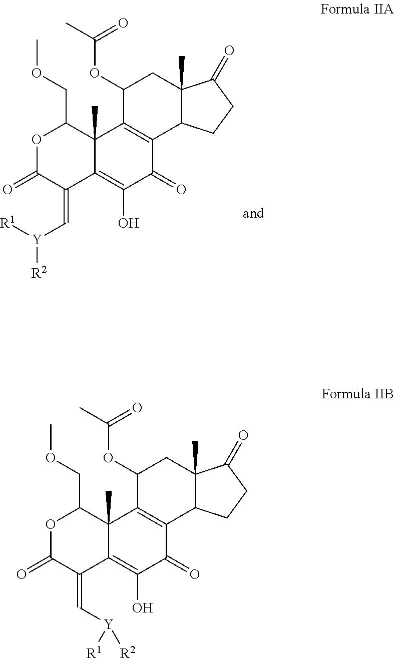

wherein Y is a heteroatom selected from nitrogen and sulfur and R¹ and R² are independently selected from an unsaturated alkyl, cyclic alkyl, or R¹ and R² together with Y form a heterocycle.

In some instances the castration resistant prostate cancer is recurrent. In other instances, the castration resistant prostate cancer is metastatic. In further instances, the castration resistant prostate cancer is unresectable.

In some embodiments of the methods provided herein, administering of the compound is by injection, transdermal, nasal, pulmonary, rectal, buccal, ocular, otic, local, topical, or oral delivery. In certain instances, injection is intramuscular, intravenous, subcutaneous, intranodal, intratumoral, intracisternal, intraperitoneal, or intradermal.

In certain embodiments, the compound is administered orally. In certain embodiments, the compound is administered in a capsule form. In certain embodiments, the compound administered in about 0.1 to about 12 mg. In certain instances, the compound is administered daily. In some instances, the compound is administered to the subject in a fasted state. In other instances, the compound is administered to the subject in a fed state.

In some embodiments, the administration is over a period of time selected from the group consisting of at least about 3 weeks, at least about 6 weeks, at least about 8 weeks, at least about 12 weeks, at least about 16 weeks, at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 68 weeks, at least about 72 weeks, at least about 90 weeks, at least about 100 weeks, at least about 110 weeks, and at least about 120 weeks.

In further embodiments of the methods provided herein, the compound is provided in a kit. In yet further embodiments, the methods provided herein further comprise an additional anti-cancer therapy. In yet further embodiments, the methods provided herein further comprise an anti-androgen. In yet further embodiments, the methods provided herein further comprise a gonadotropin-releasing hormone agonist. In yet further embodiments, the methods provided herein further comprise an anti-emetic, anti-diarrheal or both. In yet further embodiments, the methods provided herein further comprise a corticosteroid.

In some embodiments of the methods provided herein, the subject is preselected for having completed first-line anti-cancer therapy. In certain instances, the first-line anti-cancer therapy is surgery, radiation, chemotherapy, immunotherapy and/or hormone therapy.

In some embodiments of the methods provided herein, the subject is preselected for not having prior anti-cancer therapy with a PI-3 kinase inhibitor. In other embodiments, the subject is preselected for not having other active malignancies. In yet other embodiments, the subject is preselected for not having uncontrolled diabetes mellitus. In further embodiments, the subject is preselected for not being positive for human immunodeficiency virus (HIV).

In other embodiments, subject is preselected for sensitivity to administration of the compound. In certain instances, preselection is by assessment of genetic mutations in PI-3 kinase, PTEN, EGFRvIII and/or K-ras genes.

In other embodiments of the methods provided herein, the methods further comprise evaluating the treated subject, wherein the evaluation comprises determining at least one of: ((a) tumor size, (b) tumor location, (c) nodal stage, (d) growth rate of the cancer, (e) survival rate of the subject, (f) changes in the subject's cancer symptoms, (g) changes in the subject's Prostate Specific Antigen (PSA) concentration, (h) changes in the subject's PSA concentration doubling rate, (i) changes in the subject's biomarkers, or (i) changes in the subject's quality of life.

In some embodiments of the methods provided herein, the compound, suitable for treatment of castration resistant prostate cancer, is

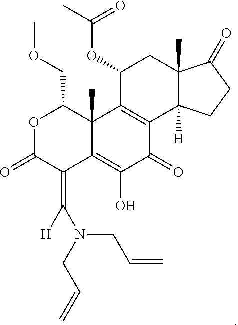 [SMILES: C=CCN(/C=C1/C(=O)O[C@H](COC)[C@@]2(C)C1=C(O)C(=O)C1=C2[C@H](OC(C)=O)C[C@]2(C)C(=O)CC[C@@H]12)CC=C]

In other embodiments of the methods provided herein, the compound, suitable for treatment of castration resistant prostate cancer, is

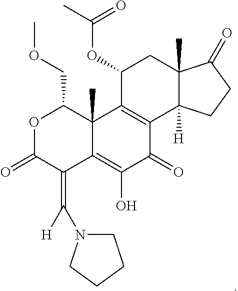 [SMILES: COC[C@H]1OC(=O)/C(=C/N2CCCC2)C2=C(O)C(=O)C3=C([C@H](OC(C)=O)C[C@]4(C)C(=O)CC[C@@H]34)[C@]21C]

In another aspect, provided herein are methods for reducing castration resistant prostate cancer tumor size in a human subject diagnosed with a castration resistant prostate cancer comprising administering to the subject a compound selected from

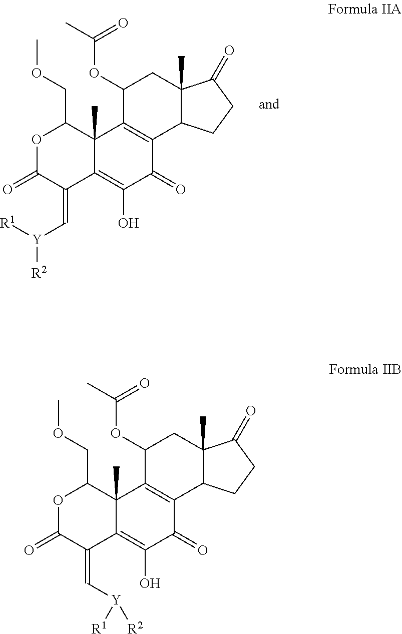

wherein Y is a heteroatom selected from nitrogen and sulfur and R¹ and R² are independently selected from an unsaturated alkyl, cyclic alkyl, or R¹ and R² together with Y form a heterocycle.

In yet another aspect, provided herein are methods for improving or maintaining the quality of life of a human subject diagnosed with a castration resistant prostate cancer comprising administering to the subject a compound selected from

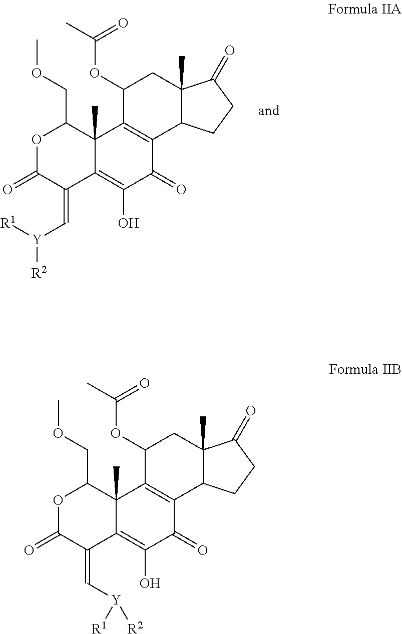

wherein Y is a heteroatom selected from nitrogen and sulfur and R¹ and R² are independently selected from an unsaturated alkyl, cyclic alkyl, or R¹ and R² together with Y form a heterocycle.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the dosing schedule for continuous dosing of PX-866 in a human clinical trial.

FIG. 2 describes certain patient characteristics in a human clinical trial for testing efficacy of PX-866 in treatment of cancer.

FIG. 3 describes certain adverse events associated with intermittent dosing of PX-866 in a human clinical trial.

FIG. 4 describes certain adverse events associated with continuous dosing of PX-866 in a human clinical trial.

FIG. 5 describes response to intermittent and continuous dosing of PX-866 in a human clinical trial.

FIG. 6 describes certain evaluable patients with stable disease following treatment with PX-866 in a human clinical trial.

FIG. 7 describes pharmacokinetics of PX-866 administration in a human clinical trial.

### DETAILED DESCRIPTION OF THE INVENTION

The PI-3 kinases are a family of related enzymes that are capable of phosphorylating the 3 position hydroxyl group of the inositol ring of phosphatidylinositol. They are linked to a diverse list of cellular functions, including cell growth, proliferation, differentiation, motility, survival and intracellular trafficking. Many of these functions relate to the ability of the PI-3 kinases to activate the protein kinase B (Akt). Genetic and pharmacological inactivation of the p110δ isoform of the PI-3 kinase has revealed this enzyme to be important for the function of T cells, B cell, mast cells and neutrophils. Hence, p110δ is considered to be a promising target for drugs that aim to prevent or treat inflammation and autoimmunity and transplant rejection. Recent evidence has shown that the gene encoding the p110α isoform of the PI-3 kinase is mutated in a range of human cancers. For example, mutation of p110α which leads to over-expression of the kinase is found in human lung cancer. PI-3 kinase activity is also found to be elevated in ovarian, head and neck, urinary tract, colon and cervical cancers. Further, a phosphate (PtdIns(3,4,5)P₃) which antagonizes PI-3 kinase activity is absent or mutated in a variety of human cancers, including advanced prostate, endometrial, renal, glial, melanoma, and small cell lung cancers. Thus, inhibition of PI-3 kinase activity provides treatment of certain human cancers.

Accordingly provided herein are certain dosing schedules and/or treatment regimens for use of PI-3 kinase inhibitors for treatment of various cancers including and not limited to solid tumors, carcinomas, myelomas, hematological cancers (e.g., leukemias, lymphomas) and/or mixed types of cancers in humans. Cancers treatable by methods described herein include, but are not limited to, breast cancer, lung cancer, head and neck cancer, brain cancer, abdominal cancer, colon cancer, colorectal cancer, esophageal cancer, gastrointestinal cancer, glioma, liver cancer, tongue cancer, neuroblastoma, osteosarcoma, ovarian cancer, renal cancer, pancreatic cancer, retinoblastoma, Wilm's tumor, multiple myeloma, skin cancer, lymphoma, leukemia, blood cancer, anaplastic thyroid tumor, sarcoma of the skin, melanoma, adenocystic tumor, hepatoid tumor, non-small cell lung cancer, chondrosarcoma, pancreatic islet cell tumor, prostate cancer, ovarian cancer, and/or carcinomas including but not limited to squamous cell carcinoma of the head and neck, colorectal carcinoma, glioblastoma, cervical carcinoma, endometrial carcinoma, gastric carcinoma, pancreatic carcinoma and breast carcinoma.

Phosphatidylinositol-3-kinases (PI-3Ks)

Phosphatidylinositol-3-kinases (PI-3Ks) are a family of intracellular lipid kinases that play a critical role in transmitting signals from cell surface receptors on the plasma membrane to downstream signaling intermediates. PI-3Ks are linked to a diverse list of cellular functions, including cell growth, proliferation, differentiation, motility, survival and intracellular trafficking. There are 3 classes of PI-3K (Class I, II and III) which are classified based upon their structure and substrate specificity. Class I PI-3K are heterodimers formed by a regulatory subunit and a catalytic p110 subunit that phosphorylate membrane-associated phosphatidylinositol 4,5-bisphosphate (PIP2) to form phosphatidylinositol 3,4,5-trisphosphate (PIP3). PIP3 binds to the serine protein kinase AKT, which is reportedly the primary effector of PI-3K, triggering activation of downstream signaling intermediates, including mammalian target of rapamycin (mTOR), with subsequent effects on cell growth and metabolism, survival, and proliferation, as well as angiogenesis. The tumor suppressor gene phosphatase and tensin homolog (PTEN) reportedly counteracts the activity of Class I PI-3K by dephosphorylating PIP3 back to PIP2. PI-3K activation reportedly affects other AKT-independent pathways including Bruton tyrosine kinase and Tec family kinases, serum and glucocorticoid regulated kinases, and regulators of GTPases, although the role of these pathways is less well defined.

Class I PI-3K is further divided into Class I_{A} and Class I_{B} subfamilies. Class I_{A} PI-3K are formed by a regulatory p85 subunit (PIK3R1) and a catalytic p110 subunit that are primarily activated by receptor tyrosine kinases such as epidermal growth factor receptor (EGFR), insulin-like growth factor (IGF), platelet-derived growth factor (PDGF) and Her2/neu. Several isoforms exist for each subunit, including α, β, γ and δ isoforms of p110. The α and β isoforms are expressed ubiquitously, whereas expression of the δ isoform is restricted to leukocytes. Class I_{B} PI-3K are composed of a p110 subunit and a p101 regulatory subunit. Class I_{B} PI-3K are activated by G protein-coupled receptors. The best characterized Class I_{B} PI-3K contains the gamma isoform of p110, and is expressed primarily in leukocytes, as well as heart, pancreas, skeletal muscle, and liver.

Increased signaling through Class I_{A} PI-3Ks has been implicated in many different forms of cancer. Cancers in which PI-3K pathway abnormalities have been identified include non-small cell lung cancer (NSCLC), breast carcinoma, ovarian carcinoma, endometrial carcinoma, prostate carcinoma, squamous cell carcinoma of the head and neck (SCCHN), cervical cancer, castration resistant prostate cancer, melanoma, and colorectal carcinoma. PI-3Ks are also contemplated in other cancers. Reported mechanisms which lead to increased signaling through the PI-3K pathway include increased receptor tyrosine kinase (RTK) activity, activating mutations in the p110 α isoform, mutations in the p85 subunit, and mutations and deletions in PTEN. Amplification of the PIK3CA gene has also been observed in a number of tumors, including squamous cell carcinomas of the lung and head and neck, although this observation has not yet been linked directly to increased PI-3K activity.

### PI-3Ks and Glioblastoma Multiforme

The response to treatment and survival of patients with glioblastoma has been shown to depend upon molecular markers. The most widely noted of these is methyl-guanine-methyl-transferase (MGMT) promoter methylation. MGMT is a DNA repair enzyme that removes methyl groups from guanine. Temozolomide is an oral alkylating agent that causes cell death by methylation of guanine bases in tumor cell DNA. It is contemplated that methylation of the MGMT promoter prevents transcription of MGMT, thereby impairing DNA repair and allowing TMZ to exert its effects on DNA.

In addition to MGMT, glioblastoma exhibits multiple genetic changes relevant to the PI-3 Kinase pathway, including alterations in the epidermal growth factor receptor (EGFR), the phosphatase and tensin homologue (PTEN) tumor suppressor and in PI-3 kinase itself. These pathways impact cellular proliferation, motility, and survival through the phosphatidylinositol-3-kinase (PI-3K) signaling pathway. EGFR mutations confer ligand-independent constitutively active isoforms, and of these, two-thirds have a deletion known as the EGFRvIII mutation that is associated with a poor prognosis. It has been reported that EGFRvIII strongly and persistently activates the PI-3K pathway.

PTEN is located at 10q23.3 and is commonly lost with chromosome 10q in glioblastoma, with approximately 58-74% of glioblastoma demonstrating loss of heterozygosity at this locus. The normal function of PTEN is to inhibit the PI-3K pathway, and loss of PTEN activity is a poor prognostic factor in GBM. The simultaneous presence in glioblastoma cells of mutant EGFR and PTEN has been associated with responsiveness to EGFR inhibitors, however, subsequent studies of EGFR inhibitors failed to corroborate these initial findings.

Finally, somatic mutations within the catalytic and regulatory subunits (PIK3CA and PIK3R1, respectively) of the PI-3K complex are also common within glioblastoma and allow for constitutive activation of the PI-3K pathway.

### PI-3Ks and Castration Resistant Prostate Cancer

It is contemplated that castration resistant prostate cancer cells survive in an environment characterized by low levels of circulating androgens by invoking continued androgen receptor signaling via alternative pathways, androgen-independent mechanisms, and/or a combination of the two. Continued androgen receptor signaling include up-regulation of the expression and copy number of the androgen receptor to enhance sensitivity to low levels of androgens and increasing the expression of enzymes involved in processing, import, and synthesis of androgens such as cytochrome C17α-hydroxylase/C_{17,20}-lyase (CYP17), an enzyme involved androgen production in the adrenals, testes, and prostate.

Androgen-independent mechanisms include activation of the androgen receptor via receptor tyrosine kinases, such as the epidermal growth factor receptor (EGFR) and downstream effectors in the PI-3 kinase pathway including the phosphatase and tensin homologue (PTEN) tumor suppressor, PI-3 kinase itself. Additional androgen-independent mechanisms include the MAP kinase pathway. It is contemplated that these pathways contribute to cellular proliferation, motility, and survival through various mechanisms including phosphorylation of the androgen receptor to allow nuclear localization and transcription and/or activation of other transcription factors independent of the androgen receptor.

In many occurrences of castration resistant prostate cancer, the mutations and disregulations in the PI-3 kinase/AKT pathway have been observed. Homozygous and heterozygous deletions of PTEN have been observed frequently (up to 60%) and contemplated to be increased in metastases. Deletions have been associated with poor patient outcomes and associated with ETS gene alterations. An estimated 30% of patients harbor gain of function mutations of the catalytic subunit of PI-3 kinase, PIK3CA, however activating mutations or amplification of AKT is less frequent. The resultant activation of the PI3K/AKT pathway leads to downstream signaling promoting survival, proliferation and angiogenesis but also has been associated with ligand independent activation and/or hypersensitization of androgen receptor signaling through a variety of mechanisms including FKHR, FKHRL1, NKκβ, Wnt/β-catenin and mTOR.

### Wortmannin Analogs

Wortmannin is a naturally occurring compound isolated from culture broths of fungal strains, Penicillium wortmannin, Talaromyces wortmannin, Penicillium Funiculosum and related micro-organisms. Wortmannin irreversibly inhibits PI-3K through covalent interaction with a specific lysine on the kinase: Lys⁸⁰² of the ATP binding pocket of the catalytic site of the p110α isoform or Lys⁸⁸³ of the p110γ isoform. Most isoforms of PI-3K, such as p110α, p110β, p110δ and p110γ for example, are inhibited equally by wortmannin. Wortmannin demonstrates liver and hematologic toxicity, however, and is a biologically unstable molecule. Samples stored as aqueous solutions at either 37° C. or 0° C. at neutral pH are subject to decomposition by hydrolytic opening of the furan ring. It has been shown that the electrophilicity of the furan ring is central to the inhibitory activity of wortmannin. The irreversible inhibition of PI-3K occurs by formation of an enamine following the attack of the active lysine of the kinase on the furan ring at position C(20) of wortmannin. Decomposition of wortmannin interferes with its inhibitory activity on PI-3Ks. Although wortmannin is a nanomolar inhibitor of PI-3K, its instability and toxicity to the liver results in variable activity in animal models. Wortmannin analogs have been contemplated and described that improve toxicity and stability of the base wortmannin compound.

In some embodiments, wortmannin analogs suitable for therapies described herein include compounds of Formula IA or IB:

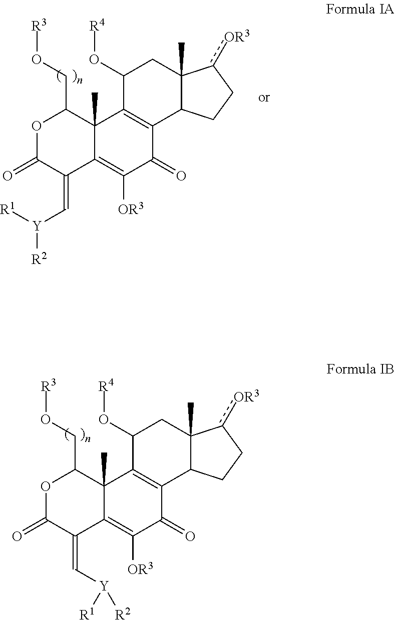

wherein:
— is an optional bond;
n is 1-6;
Y is a heteroatom;
R¹ and R² are independently selected from an unsaturated alkyl, non-linear alkyl, cyclic alkyl, and substituted alkyl or R¹ and R² together with the atom to which they are attached form a heterocycloalkyl group;
R³ is absent, H, or C₁-C₆ substituted or unsubstituted alkyl;
R⁴ is (C═O)R⁵, (C═O)OR⁵, (S═O)R⁵, (SO₂)R⁵, (PO₃)R⁵, (C═O)NR⁵R⁶;
R⁵ is substituted or unsubstituted C₁-C₆ alkyl; and
R⁶ is substituted or unsubstituted C₁-C₆ alkyl.

In some embodiments, wortmannin analogs suitable for therapies described herein include compounds of Formula IIA or IIB:

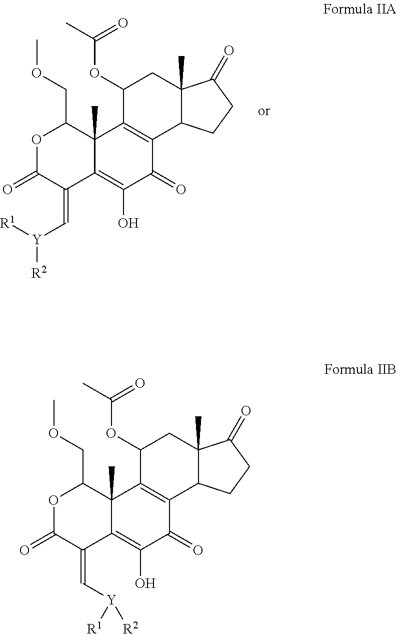

wherein Y is a heteroatom and R¹ and R² are independently selected from an unsaturated alkyl, non-linear alkyl, cyclic alkyl, and substituted alkyl or R¹ and R² together with Y form a heterocycle.

In certain embodiments of compounds of formula IIA or IIB, Y is a heteroatom selected from nitrogen and sulfur and R¹ and R² are independently selected from an unsaturated alkyl, cyclic alkyl, or R¹ and R² together with Y form a_heterocycle.

In further embodiments, a wortmannin analog is Acetic acid 4-diallylaminomethylene-6-hydroxy-1-α-methoxymethyl-10β,13β-dimethyl-3,7,17-trioxo-1,3,4,7,10,11β,12,13,14α,15,16,17-dodecahydro-2-oxa-cyclopenta[α]phenanthren-11-yl ester (PX-866) having the structure,

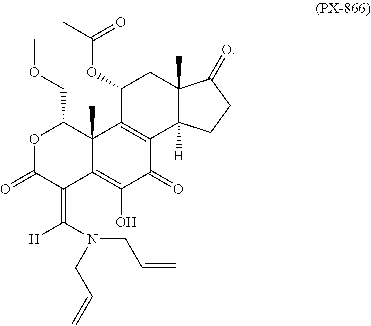 [SMILES: C=CCN(/C=C1/C(=O)O[C@H](COC)[C@@]2(C)C1=C(O)C(=O)C1=C2[C@H](OC(C)=O)C[C@]2(C)C(=O)CC[C@@H]12)CC=C]

In yet further embodiments, a wortmannin analog is Acetic acid 6-hydroxy-1α-methoxymethyl-10β,13β-dimethyl-3,7,17-trioxo-4-pyrrolidin-1-methylene-1,3,4,7,10,11β,12,13,14α,15,16,17-dodecahydro-2-oxa-cyclopenta[α]phenanthren-11-yl (PX-867) having the structure,

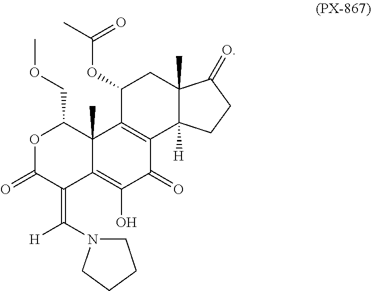 [SMILES: COC[C@H]1OC(=O)/C(=C/N2CCCC2)C2=C(O)C(=O)C3=C([C@H](OC(C)=O)C[C@]4(C)C(=O)CC[C@@H]34)[C@]21C]

In additional embodiments, wortmannin analogs suitable for therapies described herein include compounds selected from, but not limited to, PX-868, PX-870, PX-871, PX-880, PX-881, PX-882, PX-889, PX-890, DJM2-170, DJM2-171, DJM2-177, DJM2-181 and combinations thereof. In some embodiments, wortmannin analogs suitable for therapies described herein include compounds described in GB Pat. No. 2302021, which compounds are incorporated herein by reference.

### Further Forms of Wortmannin Analogs

In the scope of the embodiments, wortmannin analogs include further forms of the compounds described herein such as pharmaceutically acceptable salts, solvates (including hydrates), amorphous phases, partially crystalline and crystalline forms (including all polymorphs), prodrugs, metabolites, N-oxides, isotopically-labeled and stereo-isomers. Wortmannin analogs can be prepared as a pharmaceutically acceptable salts formed when an acidic proton present in the parent compound either is replaced by a metal ion, for example an alkali metal ion, an alkaline earth ion, or an aluminum ion; or coordinates with an organic base. In addition, the salt forms of the disclosed compounds can be prepared using salts of the starting materials or intermediates.

In some of the embodiments described herein, wortmannin analogs can be prepared as a pharmaceutically acceptable acid addition salt (which is a type of a pharmaceutically acceptable salt) by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, Q-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

Alternatively, in some of the embodiments described herein, wortmannin analogs can be prepared as a pharmaceutically acceptable base addition salts (which is a type of a pharmaceutically acceptable salt) by reacting the free acid form of the compound with a pharmaceutically acceptable inorganic or organic base, including, but not limited to organic bases such as ethanolamine, diethanolamine, triethanolamine, tromethamine, N-methylglucamine, and the like and inorganic bases such as aluminum hydroxide, calcium hydroxide, potassium hydroxide, sodium carbonate, sodium hydroxide, and the like.

It should be understood that a reference to a pharmaceutically acceptable salt includes the solvent addition forms or crystal forms thereof, particularly solvates or polymorphs. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of wortmannin analogs can be conveniently prepared or formed during the processes described herein. By way of example only, hydrates of wortmannin analogs can be conveniently prepared by recrystallization from an aqueous/organic solvent mixture, using organic solvents including, but not limited to, dioxane, toluene, alkyl acetate, anisole, tetrahydrofuran or methanol. In addition, the compounds provided herein can exist in unsolvated as well as solvated forms. In general, the solvated forms are considered equivalent to the unsolvated forms for the purposes of the compounds and methods provided herein.

In some of the embodiments described herein, wortmannin analogs include crystalline forms, also known as polymorphs. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

In some of the embodiments described herein, wortmannin analogs in unoxidized form can be prepared from N-oxides of compounds of Formula (I) by treating with a reducing agent, such as, but not limited to, sulfur, sulfur dioxide, triphenyl phosphine, lithium borohydride, sodium borohydride, phosphorus trichloride, tribromide, or the like in a suitable inert organic solvent, such as, but not limited to, acetonitrile, ethanol, aqueous dioxane, or the like at 0 to 80° C.

In some embodiments, wortmannin analogs are isotopically-labeled, which are identical to those recited in the various formulae and structures presented herein, but for the fact that one or more atoms are replaced by an atom having an atomic mass or mass number different from the atomic mass or mass number usually found in nature. In some embodiments, one or more hydrogen atoms are replaced with deuterium. In some embodiments, metabolic sites on the compounds described herein are deuterated. In some embodiments, substitution with deuterium affords certain therapeutic advantages resulting from greater metabolic stability, such as, for example, increased in vivo half-life or reduced dosage requirements.

In some of the embodiments described herein, wortmannin analogs can be prepared as prodrugs. Prodrugs are generally drug precursors that, following administration to a subject and subsequent absorption, are converted to an active, or a more active species via some process, such as conversion by a metabolic pathway. Some prodrugs have a chemical group present on the prodrug that renders it less active and/or confers solubility or some other property to the drug. Once the chemical group has been cleaved and/or modified from the prodrug the active drug is generated. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a wortmannin analog which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

In some of the embodiments described herein, wortmannin analogs are metabolites. A “metabolite” of a wortmannin analog disclosed herein is a derivative of that wortmannin analog that is formed when the wortmannin analog is metabolized. The term “active metabolite” refers to a biologically active derivative of a wortmannin analog that is formed when the wortmannin analog is metabolized (biotransformed). The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a wortmannin analog. For example, cytochrome P450 catalyzes a variety of oxidative and reductive reactions while uridine diphosphate glucuronyltransferases (UGT) catalyze the transfer of an activated glucuronic-acid molecule to aromatic alcohols, aliphatic alcohols, carboxylic acids, amines and free sulphydryl groups (e.g. conjugation reactions). Further information on metabolism is available in The Pharmacological Basis of Therapeutics, 9th Edition, McGraw-Hill (1996). In one embodiment, metabolites of the compounds disclosed herein are identified either by administration of compounds to a host and analysis of tissue samples from the host, or by incubation of compounds with hepatic cells in vitro and analysis of the resulting compounds.

Metabolites of wortmannin analogs, in some embodiments described herein, include, but are not limited to, metabolites resulting from first pass metabolism. In some embodiments, the metabolite is a 17-hydroxy (17-OH) derivative of a wortmannin analog. In some embodiments, the metabolite is a derivative of PX-866. In other embodiments, the metabolite is a derivative of PX-867.

In some instances a metabolite of PX-866 has the following structural formula:

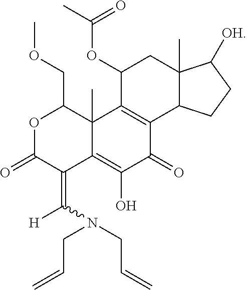 [SMILES: C=CCN(C=C1C(=O)OC(COC)C2(C)C1=C(O)C(=O)C1=C2C(OC(C)=O)CC2(C)C(O)CCC12)CC=C]

In other instances a metabolite of PX-867 has the following structural formula:

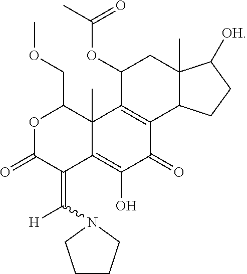 [SMILES: COCC1OC(=O)C(=CN2CCCC2)C2=C(O)C(=O)C3=C(C(OC(C)=O)CC4(C)C(O)CCC34)C21C]

In further embodiments, a metabolite of a wortmannin analog is a 11,17-hydroxy (11,17-OH) derivative of a wortmannin analog.

In some instances a metabolite of PX-866 has the following structural formula:

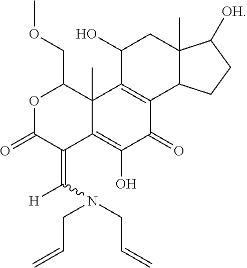 [SMILES: C=CCN(C=C1C(=O)OC(COC)C2(C)C1=C(O)C(=O)C1=C2C(O)CC2(C)C(O)CCC12)CC=C]

In other instances a metabolite of PX-867 has the following structural formula:

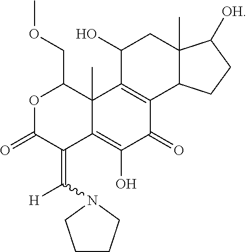 [SMILES: COCC1OC(=O)C(=CN2CCCC2)C2=C(O)C(=O)C3=C(C(O)CC4(C)C(O)CCC34)C21C]

PX-866 is an pan-isoform inhibitor of Class I P1-3K that covalently binds to ATP binding site of the p110 catalytic subunit. Described herein are studies that illustrate rapid metabolism of PX-866 to a 17-hydroxy PX-866 derivative. The 17-hydroxy PX-866 metabolite has a 2-5 fold increase in potency in cell proliferation assays versus p110α and p110β isoforms. For example, in cell based assays, potency of the 17-hydroxy metabolite is p110α IC50 14 nM vs 39 nM for the parent compound (PX-866), potency of the 17-hydroxy metabolite is p110β IC50 57 nM vs. 88 nM for the parent compound (PX-866).

IC50 nM

| Target | PX-866 | 17-OH PX-866 |
|---|---|---|
| PIK3CA | 39 | 14 |
| PIK3CB | 88 | 57 |
| PIK3CD | 124 | 131 |
| PIK3CG | 198 | 148 |

### Synthesis of Wortmannin Analogs

Wortmannin analogs described herein may be synthesized using standard synthetic techniques known to those of skill in the art or using methods known in the art in combination with methods described herein. In additions, solvents, temperatures and other reaction conditions presented herein may vary according to the practice and knowledge of those of skill in the art.

The starting material used for the synthesis of wortmannin analogs described herein can be obtained from commercial sources, such as Aldrich Chemical Co. (Milwaukee, Wis.), Sigma Chemical Co. (St. Louis, Mo.), or the starting materials can be synthesized. The wortmannin analogs described herein, and other related compounds having different substituents can be synthesized using techniques and materials known to those of skill in the art, such as described, for example, in March, ADVANCED ORGANIC CHEMISTRY 4th Ed., (Wiley 1992); Carey and Sundberg, ADVANCED ORGANIC CHEMISTRY 4th Ed., Vols. A and B (Plenum 2000, 2001), and Green and Wuts, PROTECTIVE GROUPS IN ORGANIC SYNTHESIS 3rd Ed., (Wiley 1999) (all of which are incorporated by reference in their entirety). General methods for the preparation of wortmannin analogs as disclosed herein may be derived from known reactions in the field, and the reactions may be modified by the use of appropriate reagents and conditions, as would be recognized by the skilled person, for the introduction of the various moieties found in the formulae as provided herein.

Additional synthesis methods and schemes for the wortmannin analogs described herein can be found in, for example, U.S. Pat. No. 5,480,906, U.S. Pat. No. 7,335,679, and U.S. Patent Appl. Pub. No. 2007/0191466, each of which is incorporated herein by reference for synthesis of wortmannin analogs.

### Methods

Provided herein, in some embodiments, are methods of treatment of cancers comprising administration of wortmannin analogs described herein (e.g., compounds of Formula IA, IB, IIA or IIB or any other PI-3 kinase inhibitor and/or wortmannin analog described herein) to individuals in need thereof. In some embodiments, wortmannin analogs are administered to individuals in need thereof in a continuous dosing regimen as described herein. In some embodiments, wortmannin analogs are administered to individuals in need thereof in an intermittent dosing regimen as described herein. In some embodiments, provided herein are method of treatment of cancers comprising administration of wortmannin analogs (e.g., a compound of Formula IA, Formula IB, Formula IIA, Formula IIB, PX-866 or PX-867) to individuals in need thereof. In some embodiments, wortmannin analogs (e.g., compounds of Formula IA, Formula IB, Formula IIA or Formula IIB) are irreversible PI-3 kinase inhibitors.

In some of such embodiments, the use of wortmannin analogs (e.g., a compound of Formula IA, Formula IB, Formula IIA, Formula IIB, PX-866 or PX-867) that are covalent modifiers of PI-3 kinase allow for chronic dosing at low doses of a chemotherapeutic (e.g., PX-866 and/or metabolites thereof) in continuous dosing regimens described herein while avoiding side-effects associated with currently approved chemotherapeutics. In some of such embodiments, the use of wortmannin analogs (e.g., a compound of Formula IA, Formula IB, Formula IIA, Formula IIB, PX-866 or PX-867) that are covalent modifiers of PI-3 kinase in low doses in continuous dosing regimens reduces or ameliorates side-effects such as elevation in ALT/AST and/or proteinuria that occur upon chronic dosing of currently approved chemotherapeutics.

In some embodiments, the use of wortmannin analogs (e.g., a compound of Formula IA, Formula IB, Formula IIA, Formula IIB, PX-866 or PX-867) that are covalent modifiers of PI-3 kinase allow for the use of low doses of a chemotherapeutic (e.g., PX-866 and/or metabolites thereof) in intermittent dosing regimens while avoiding side-effects associated with currently approved chemotherapeutics.

In one embodiment, provided herein is a method of treatment of cancers comprising administration of a wortmannin analog (e.g., a compound of Formula IA, Formula IB, Formula IIA, Formula IIB, PX-866 or PX-867) to an individual in need thereof at a dose and frequency of administration sufficient to result in a plasma concentration of the wortmannin analog (e.g., a compound of Formula IA, Formula IB, Formula IIA, Formula IIB, PX-866 or PX-867) and/or an active metabolite thereof (e.g., 17-hydroxy PX-866, 17-hydroxy PX-867) between about 250 pg/mL and about 5000 pg/mL (peak) within about 1-8 hours of administration of the wortmannin analog. In one embodiment, provided herein is a method of treatment of cancers comprising administration of a wortmannin analog (e.g., a compound of Formula IA, Formula IB, Formula IIA, Formula IIB, PX-866 or PX-867) to an individual in need thereof at a dose and frequency of administration sufficient to result in a plasma concentration of the wortmannin analog (e.g., a compound of Formula IA, Formula IB, Formula IIA, Formula IIB, PX-866 or PX-867) and/or an active metabolite thereof (e.g., 17-hydroxy PX-866, 17-hydroxy PX-867) between about 500 pg/mL and about 4000 pg/mL (peak) within about 1-8 hours of administration of the wortmannin analog. In one embodiment, provided herein is a method of treatment of cancers comprising administration of a wortmannin analog (e.g., a compound of Formula IA, Formula IB, Formula IIA, Formula IIB, PX-866 or PX-867) to an individual in need thereof at a dose and frequency of administration sufficient to result in a plasma concentration of the wortmannin analog (e.g., a compound of Formula IA, Formula IB, Formula IIA, Formula IIB, PX-866 or PX-867) and/or an active metabolite thereof (e.g., 17-hydroxy PX-866, 17-hydroxy PX-867) between about 500 pg/mL and about 2500 pg/mL (peak) within about 1-3 hours of administration of the wortmannin analog. In one embodiment, provided herein is a method of treatment of cancers comprising administration of a wortmannin analog (e.g., a compound of Formula IA, Formula IB, Formula IIA, Formula IIB, PX-866 or PX-867) to an individual in need thereof at a dose and frequency of administration sufficient to result in a plasma concentration of the wortmannin analog (e.g., a compound of Formula IA, Formula IB, Formula IIA, Formula IIB, PX-866 or PX-867) and/or an active metabolite thereof (e.g., 17-hydroxy PX-866, 17-hydroxy PX-867) between about 600 pg/mL and about 2000 pg/mL (peak) within about 1-3 hours of administration of the wortmannin analog. In one embodiment, provided herein is a method of treatment of cancers comprising administration of a wortmannin analog (e.g., a compound of Formula IA, Formula IB, Formula IIA, Formula IIB, PX-866 or PX-867) to an individual in need thereof at a dose and frequency of administration sufficient to result in a plasma concentration of the wortmannin analog (e.g., a compound of Formula IA, Formula IB, Formula IIA, Formula IIB, PX-866 or PX-867) and/or an active metabolite thereof (e.g., 17-hydroxy PX-866, 17-hydroxy PX-867) between about 750 pg/mL and about 1900 pg/mL (peak) within about 1-3 hours of administration of the wortmannin analog. In one embodiment, provided herein is a method of treatment of cancers comprising administration of a wortmannin analog (e.g., a compound of Formula IA, Formula IB, Formula IIA, Formula IIB, PX-866 or PX-867) to an individual in need thereof at a dose and frequency of administration sufficient to result in a plasma concentration of the wortmannin analog (e.g., a compound of Formula IA, Formula IB, Formula IIA, Formula IIB, PX-866 or PX-867) and/or an active metabolite thereof (e.g., 17-hydroxy PX-866, 17-hydroxy PX-867) between about 750 pg/mL and about 1750 pg/mL (peak) within about 1-3 hours of administration of the wortmannin analog. In some specific embodiments, for any of the aforementioned embodiments, the wortmannin analog is PX-866 and/or an active metabolite thereof (e.g., 17-hydroxy PX-866). In some specific embodiments, for any of the aforementioned embodiments, the wortmannin analog is a 17-hydroxy metabolite of PX-866.

In one embodiment, provided herein is a method of treatment of cancers comprising administration of a wortmannin analog (e.g., a compound of Formula IA, Formula IB, Formula IIA, Formula IIB, PX-866 or PX-867) to an individual in need thereof at a dose and frequency of administration sufficient to reduce or alleviate side-effects associated with long-term and/or chronic and/or continuous dosing. In some embodiments, the wortmannin analog (e.g., a compound of Formula IA, Formula IB, Formula IIA, Formula IIB, PX-866 or PX-867) is administered to the subject in an amount of from about 0.01 mg to about 200 mg per day. In some embodiments, the wortmannin analog (e.g., a compound of Formula IA, Formula IB, Formula IIA, Formula IIB, PX-866 or PX-867) is administered to the subject in an amount of from about 0.01 mg to about 100 mg per day. In some embodiments, the wortmannin analog (e.g., a compound of Formula IA, Formula IB, Formula IIA, Formula IIB, PX-866 or PX-867) is administered to the subject in a low dose in an amount of from about 0.01 mg to about 50 mg per day. In some embodiments, the wortmannin analog (e.g., a compound of Formula IA, Formula IB, Formula IIA, Formula IIB, PX-866 or PX-867) is administered to the subject in a low dose in an amount of from about 0.1 mg to about 25 mg per day. In some embodiments, the wortmannin analog (e.g., a compound of Formula IA, Formula IB, Formula IIA, Formula IIB, PX-866 or PX-867) is administered to the subject in a low dose in an amount of from about 0.5 to about 16 mg per day. In some embodiments, the wortmannin analog (e.g., a compound of Formula IA, Formula IB, Formula IIA, Formula IIB, PX-866 or PX-867) is administered to the subjects in a low dose in an amount of between about 1 to about 14 mg per day. In some embodiments, the wortmannin analog (e.g., a compound of Formula IA, Formula IB, Formula IIA, Formula IIB, PX-866 or PX-867) is administered to the subjects in a low dose in an amount of between about 2 mg to about 12 mg per day. In some embodiments, the wortmannin analog (e.g., a compound of Formula IA, Formula IB, Formula IIA, Formula IIB, PX-866 or PX-867) is administered to the subjects in an amount of between about 2 mg to about 10 mg per day. In some embodiments, the wortmannin analog (e.g., a compound of Formula IA, Formula IB, Formula IIA, Formula IIB, PX-866 or PX-867) is administered to the subjects in a low dose in an amount of between about 2 mg to about 8 mg per day. In some embodiments, the wortmannin analog (e.g., a compound of Formula IA, Formula IB, Formula IIA, Formula IIB, PX-866 or PX-867) is administered to the subjects in a low dose in an amount of between about 2 mg to about 6 mg per day. In some embodiments, the wortmannin analog (e.g., a compound of Formula IA, Formula IB, Formula IIA, Formula IIB, PX-866 or PX-867) is administered to the subjects in a low dose in an amount of between about 2 mg to about 4 mg per day. In some specific embodiments, for any of the aforementioned embodiments, the wortmannin analog is PX-866 and/or an active metabolite thereof (e.g., 17-hydroxy PX-866). In some specific embodiments, for any of the aforementioned embodiments, the wortmannin analog is a 17-hydroxy metabolite of PX-866.

In some of the above embodiments, the wortmannin analog (e.g., a compound of Formula IA, Formula IB, Formula IIA, Formula IIB, PX-866, 17-hydroxy PX-866) is administered to the subject as an intermittent dose. In some other embodiments described above, the wortmannin analog (e.g., a compound of Formula IA, Formula IB, Formula IIA, Formula IIB, PX-866, 17-hydroxy PX-866) is administered to the subject as a continuous dose.

In one embodiment, provided herein is a method of treatment of cancers comprising administration of a wortmannin analog (e.g., a compound of Formula IA, Formula IB, Formula IIA, Formula IIB, PX-866 or PX-867) to an individual in need thereof at a dose and frequency of administration sufficient to provide a plasma Cₘₐₓ of the wortmannin analog (e.g., a compound of Formula IA, Formula IB, Formula IIA, Formula IIB, PX-866 or PX-867) and/or a metabolite thereof (e.g., 17-hydroxy PX-866, 17-hydroxy PX-867) between about 250 pg/mL and about 5000 pg/mL. In one embodiment, provided herein is a method of treatment of cancers comprising administration of a wortmannin analog (e.g., a compound of Formula IA, Formula IB, Formula IIA, Formula IIB, PX-866 or PX-867) to an individual in need thereof at a dose and frequency of administration sufficient to provide a plasma Cₘₐₓ of the wortmannin analog (e.g., a compound of Formula IA, Formula IB, Formula IIA, Formula IIB, PX-866 or PX-867) and/or a metabolite thereof (e.g., 17-hydroxy PX-866, 17-hydroxy PX-867) between about 500 pg/mL and about 4000 pg/mL. In one embodiment, provided herein is a method of treatment of cancers comprising administration of a wortmannin analog (e.g., a compound of Formula IA, Formula IB, Formula IIA, Formula IIB, PX-866 or PX-867) to an individual in need thereof at a dose and frequency of administration sufficient to provide a plasma Cₘₐₓ of the wortmannin analog (e.g., a compound of Formula IA, Formula IB, Formula IIA, Formula IIB, PX-866 or PX-867) and/or a metabolite thereof (e.g., 17-hydroxy PX-866, 17-hydroxy PX-867) between about 600 pg/mL and about 3000 pg/mL. In one embodiment, provided herein is a method of treatment of cancers comprising administration of a wortmannin analog (e.g., a compound of Formula IA, Formula IB, Formula IIA, Formula IIB, PX-866 or PX-867) to an individual in need thereof at a dose and frequency of administration sufficient to provide a plasma Cₘₐₓ of the wortmannin analog (e.g., a compound of Formula IA, Formula IB, Formula IIA, Formula IIB, PX-866 or PX-867) and/or a metabolite thereof (e.g., 17-hydroxy PX-866, 17-hydroxy PX-867) between about 750 pg/mL and about 2000 pg/mL. In some specific embodiments, for any of the aforementioned embodiments, the wortmannin analog is PX-866 and/or an active metabolite thereof (e.g., 17-hydroxy PX-866). In some specific embodiments, for any of the aforementioned embodiments, the wortmannin analog is a 17-hydroxy metabolite of PX-866.

In one embodiment, provided herein is a method of treatment of cancers comprising administration of a wortmannin analog (e.g., a compound of Formula IA, Formula IB, Formula IIA, Formula IIB, PX-866 or PX-867) to an individual in need thereof at a dose and frequency of administration sufficient to provide AUC of between about 500 hr*pg/mL and about 12,000 hr*pg/mL for the wortmannin analog (e.g., a compound of Formula IA, Formula IB, Formula IIA, Formula IIB, PX-866 or PX-867) and/or a metabolite thereof (e.g., 17-hydroxy PX-866, 17-hydroxy PX-867). In one embodiment, provided herein is a method of treatment of cancers comprising administration of a wortmannin analog (e.g., a compound of Formula IA, Formula IB, Formula IIA, Formula IIB, PX-866 or PX-867) to an individual in need thereof at a dose and frequency of administration sufficient to provide AUC of between about 1000 hr*pg/mL and about 10,000 hr*pg/mL for the wortmannin analog (e.g., a compound of Formula IA, Formula IB, Formula IIA, Formula IIB, PX-866 or PX-867) and/or a metabolite thereof (e.g., 17-hydroxy PX-866, 17-hydroxy PX-867). In one embodiment, provided herein is a method of treatment of cancers comprising administration of a wortmannin analog (e.g., a compound of Formula IA, Formula IB, Formula IIA, Formula IIB, PX-866 or PX-867) to an individual in need thereof at a dose and frequency of administration sufficient to provide AUC of between about 2000 hr*pg/mL and about 8000 hr*pg/mL for the wortmannin analog (e.g., a compound of Formula IA, Formula IB, Formula IIA, Formula IIB, PX-866 or PX-867) and/or a metabolite thereof (e.g., 17-hydroxy PX-866, 17-hydroxy PX-867). In some specific embodiments, for any of the aforementioned embodiments, the wortmannin analog is PX-866 and/or an active metabolite thereof (e.g., 17-hydroxy PX-866). In some specific embodiments, for any of the aforementioned embodiments, the wortmannin analog is a 17-hydroxy metabolite of PX-866.

In one embodiment, provided herein is a method of treatment of cancers comprising administration of a wortmannin analog (e.g., a compound of Formula IA, Formula IB, Formula IIA, Formula IIB, PX-866 or PX-867) to an individual in need thereof at a dose and frequency of administration sufficient to reduce and/or alleviate incidence of proteinuria and/or elevated ALT/AST. In some specific embodiments, the wortmannin analog is PX-866 and/or an active metabolite thereof (e.g., 17-hydroxy PX-866). In some specific embodiments, the wortmannin analog is a 17-hydroxy metabolite of PX-866.

In one embodiment, provided herein is a method of treatment of cancers comprising administration of a wortmannin analog (e.g., a compound of Formula IA, Formula IB, Formula IIA, Formula IIB, PX-866 or PX-867) to an individual in need thereof at a dose and frequency of administration sufficient to result in disease stabilization (for example, a delay in disease progression and/or suppression in disease progression). In some specific embodiments, the wortmannin analog is PX-866 and/or an active metabolite thereof (e.g., 17-hydroxy PX-866). In some specific embodiments, the wortmannin analog is a 17-hydroxy metabolite of PX-866.

In one embodiment, provided herein is a method of treatment of cancers comprising administration of a wortmannin analog (e.g., a compound of Formula IA, Formula IB, Formula IIA, Formula IIB, PX-866 or PX-867) to an individual in need thereof at a dose and frequency of administration sufficient to result in disease remission. In some specific embodiments, the wortmannin analog is PX-866 and/or an active metabolite thereof (e.g., 17-hydroxy PX-866). In some specific embodiments, the wortmannin analog is a 17-hydroxy metabolite of PX-866.

In one embodiment, provided herein is a method of treatment of cancers comprising administration of a wortmannin analog (e.g., a compound of Formula IA, Formula IB, Formula IIA, Formula IIB, PX-866 or PX-867) to an individual in need thereof as a low and continuous dose. As used herein, a “low dose” or “lower dose” suitable for continuous dosing is between about 10% and about 85% of the maximal tolerated dose (MTD) of intermittent dosing and provides a therapeutic benefit to an individual in need thereof. In some embodiments, a low dose suitable for continuous dosing is between about 15% and about 85% of the MTD of intermittent dosing and provides a therapeutic benefit to an individual in need thereof. In some embodiments, a low dose suitable for continuous dosing is between about 25% and about 85% of the MTD of intermittent dosing and provides a therapeutic benefit to an individual in need thereof. In some embodiments, a low dose suitable for continuous dosing is between about 35% and about 85% of the MTD of intermittent dosing and provides a therapeutic benefit to an individual in need thereof. In some embodiments, a low dose suitable for continuous dosing is between about 50% and about 75% of the MTD of intermittent dosing and provides a therapeutic benefit to an individual in need thereof. In some embodiments, a low dose suitable for continuous dosing is between about 10% and about 60% of the MTD of intermittent dosing and provides a therapeutic benefit to an individual in need thereof. In some embodiments, a low dose suitable for continuous dosing is between about 15% and about 50% of the MTD of intermittent dosing and provides a therapeutic benefit to an individual in need thereof. In some specific embodiments, the wortmannin analog is PX-866 and/or an active metabolite thereof (e.g., 17-hydroxy PX-866). In some specific embodiments, the wortmannin analog is a 17-hydroxy metabolite of PX-866.

### Treatment of Glioblastoma

Also provided herein, in certain embodiments, are methods for treating glioblastoma in a subject with a wortmannin analog. Also provided herein are compounds, pharmaceutical compositions and medicaments comprising a wortmannin analog for use in treating a subject with glioblastoma.

In some aspects, the wortmannin analogs described herein treat various forms of glioblastoma including forms which are metastatic and/or recurrent in a subject. Glioblastoma which is metastatic is a stage where the glioblastoma spreads to other parts of the brain or throughout the body to distant tissues and organs. Glioblastoma designated as recurrent generally is defined as glioblastoma that has recurred or relapsed, usually after a period of time, after being in remission or after a tumor has visibly been eliminated. Recurrence can either be local, i.e., appearing in the same location as the original, or distant, i.e., appearing in a different part of the brain. In some embodiments, the wortmannin analogs described herein are used to treat metastatic glioblastoma in a subject. In other embodiments, the wortmannin analogs described herein are used to treat recurrent glioblastoma in a subject. In certain instances, glioblastoma treatable wortmannin analogs described herein is unresectable, or unable to be removed by surgery.

In certain aspects, the wortmannin analogs described herein treat variants or subtypes of glioblastoma in a subject. Variants or subtypes of glioblastoma include, but are not limited to, primary glioblastoma, secondary glioblastoma, gliosarcoma, multifocal GBM and gliomatosis cerebri.

In other aspects, the wortmannin analogs described herein treat precursor tumor stages that lead to glioblastoma in a subject. Precursor tumor stages include those described in the World Health Organization astrocytoma grading system. WHO Grade 1 includes low grade astrocytomas such as pilocytic astrocytomas; Grade 2 includes fibrillary or diffuse astrocytomas; and Grade 3 including anaplastic astrocytomas. Grade 4 is glioblastoma which is generally characterized as anaplastic astrocytomas surrounded by necrotizing tissue. In certain cases, hyperplastic blood vessels are present in Grade 4. In some embodiments, the wortmannin analogs described herein treat low grade astrocytomas. In other embodiments, the wortmannin analogs described herein treat fibrillary or diffuse astrocytomas. In yet other embodiments, the wortmannin analogs described herein treat anaplastic astrocytomas. In yet other embodiments, the wortmannin analogs described herein treat anaplastic astrocytomas surrounded by necrotizing tissue.

In some embodiments, the wortmannin analogs described herein are administered as a first-line or primary therapy. Other subjects suitable for treatment by the wortmannin analogs described herein include those that have completed first-line anti-cancer therapy. First-line anti-cancer therapies include chemotherapy, radiotherapy, immunotherapy, gene therapy, hormone therapy, surgery or other therapies that are capable of negatively affecting glioblastoma in a patient, such as for example, by killing glioblastoma cells, inducing apoptosis in glioblastoma cells, reducing the growth rate of glioblastoma cells, reducing the incidence or number of metastases, reducing tumor size, inhibiting tumor growth, reducing the blood supply to a tumor or cancer cells, promoting an immune response against cancer cells or a tumor, preventing or inhibiting the progression of glioblastoma, or increasing the lifespan of a subject with glioblastoma.

Chemotherapies for first-line and subsequent therapy include, but are not limited to, temozolomide, mitozolomide, dacarbazine, cisplatin (CDDP), carboplatin, procarbazine, mechlorethamine, cyclophosphamide, camptothecin, ifosfamide, melphalan, chlorambucil, busulfan, nitrosurea, dactinomycin, anthracyclines (e.g., daunorubicin, doxorubicin, epirubicin, idarubicin), bleomycin, plicomycin, mitomycin, etoposide (VP16), tamoxifen, raloxifene, estrogen receptor binding agents, docetaxel, paclitaxel, gemcitabine, navelbine, farnesyl-protein transferase inhibitors, transplatinum, 5-fluorouracil, capecitabine, vincristin, vinblastin and methotrexate, topoisomerase inhibitors (e.g., irinotecan, topotecan, camptothecin, etoposide) or any derivative related agent of the foregoing.

Radiotherapies for first-line and subsequent therapy include factors that cause DNA damage and include what are commonly known as γ-rays, X-rays, and/or the directed delivery of radioisotopes to tumor cells. Other forms of DNA damaging factors include microwaves and UV-irradiation. It is likely that all of these factors affect a broad range of damage on DNA, on the precursors of DNA, on the replication and repair of DNA, and on the assembly and maintenance of chromosomes. Dosage ranges for X-rays may range from daily doses of 50 to 200 roentgens for prolonged periods of time (e.g., 3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

Immunotherapies generally rely on the use of immune effector cells and molecules to target and destroy cancer cells. The immune effector may be, for example, a tumor antigen or an antibody specific for some marker on the surface of a tumor cell. The tumor antigen or antibody alone may serve as an effector of therapy or it may recruit other cells to actually effect cell killing. An antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. Alternatively, a tumor antigen may stimulate a subject's immune system to target the specific tumor cells using cytotoxic T cells and NK cells. Exemplary immunotherapies for glioblastoma include bevacizumab, a monoclonal antibody targeting the vascular endothelial growth factor receptor (VEGF-R).

A gene therapy includes a therapeutic polynucleotide is administered before, after, or at the same time as a combination therapy. Therapeutic genes may include an antisense version of an inducer of cellular proliferation (oncogene), an inhibitor of cellular proliferation (tumor suppressor), or an inducer of programmed cell death (pro-apoptotic gene).

Surgery of some type is performed for resectable glioblastomas. Surgery types include preventative, diagnostic or staging, curative and palliative surgery and can be performed as a first-line and subsequent therapy.

In some embodiments, the wortmannin analogs described herein are administered as a second-line therapy after a first-line therapy becomes ineffective or the glioblastoma is recurrent. In other embodiments, the wortmannin analogs described herein administered as a third-line therapy after the first- and second-line therapy fails. In further embodiments, individuals are preselected for having completed a first- or second-line therapy. In some instances, the wortmannin analogs described herein are administered to patients for whom prior DNA alkylating agent therapy has failed. In other instances, the wortmannin analogs described herein are administered to patients for whom prior temozolomide therapy has failed.

### Treatment of Castration Resistant Prostate Cancer

Also provided herein, in certain embodiments, are methods for treating castration resistant prostate cancer in a subject with a wortmannin analog. Also provided herein are compounds, pharmaceutical compositions and medicaments comprising a wortmannin analog for use in treating a subject with castration resistant prostate cancer.

In some aspects, the wortmannin analogs described herein treat various forms of castration resistant prostate cancer including forms which are metastatic and/or recurrent in a subject. Castration resistant prostate cancer which is metastatic is a stage where the castration resistant prostate cancer spreads to other parts of the body to distant tissues and organs. Castration resistant prostate cancer designated as recurrent generally is defined as castration resistant prostate cancer that has recurred or relapsed, usually after a period of time, after being in remission or after a tumor has visibly been eliminated. Recurrence can either be local, i.e., appearing in the same location as the original, or distant, i.e., appearing in a different part of the body. In some embodiments, the wortmannin analogs described herein are used to treat metastatic castration resistant prostate cancer in a subject. In other embodiments, the wortmannin analogs described herein are used to treat recurrent castration resistant prostate cancer in a subject. In certain instances, castration resistant prostate cancer treatable wortmannin analogs described herein is unresectable, or unable to be removed by surgery.

In certain aspects, the wortmannin analogs described herein treat any stage or grade of castration resistant prostate cancer in a subject. Castration resistant prostate cancer staging includes T (tumor), N (node), M (metastasis) staging (American Joint Committee on Cancer 2002) as well as commonly used Roman Numeral I-IV staging. Castration resistant prostate cancer grading includes the Gleason Grading wherein the diseased prostatic tissue is compared to normal tissue and designated a number from 1-5, with increasing numbers having lesser similarity to normal prostatic tissue. In some embodiments, the wortmannin analogs described herein treat castration resistant prostate cancer in a subject wherein T is T1-T4, N is N0-N1 and M is M0-M1 in TMN stage of the prostate cancer. In other embodiments, the wortmannin analogs described herein treat castration resistant prostate cancer in a subject wherein the prostate cancer is Stage I, Stage II, Stage III or Stage IV. In further embodiments, the wortmannin analogs described herein treat castration resistant prostate cancer in a subject wherein the prostate cancer has a Gleason Grade of 1, 2, 3, 4 or 5.

In some embodiments, the wortmannin analogs described herein are administered as a first-line or primary therapy to a subject. Other subjects suitable for treatment by the wortmannin analogs described herein include those that have completed first-line anti-cancer therapy. First-line anti-cancer therapies include chemotherapy, radiotherapy, immunotherapy, gene therapy, hormone therapy, surgery or other therapies that are capable of negatively affecting prostate cancer in a patient, such as for example, by killing prostate cancer cells, inducing apoptosis in prostate cancer cells, reducing the growth rate of prostate cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of castration resistant prostate cancer, or increasing the lifespan of a subject with castration resistant prostate cancer.

Chemotherapies for first-line and subsequent therapy include, but are not limited to, hormone modulators, androgen receptor binding agents (e.g., anti-androgens, bicalutamide, flutamide, nilutamide, MDV3100), gonadotropin-releasing hormone agonists and antagonists (e.g., leuprolide, buserelin, histrelin, goserelin, deslorelin, nafarelin, abarelix, cetrorelix, ganirelix degarelix), androgen synthesis inhibitors (abiraterone, TOK-001), temozolomide, mitozolomide, dacarbazine, cisplatin (CDDP), carboplatin, procarbazine, mechlorethamine, cyclophosphamide, camptothecin, ifosfamide, melphalan, chlorambucil, busulfan, nitrosurea, dactinomycin, anthracyclines (e.g., daunorubicin, doxorubicin, epirubicin, idarubicin), bleomycin, plicomycin, mitomycin, etoposide (VP16), tamoxifen, raloxifene, estrogen receptor binding agents, docetaxel, paclitaxel, cabazitaxol, gemcitabine, navelbine, farnesyl-protein transferase inhibitors, transplatinum, 5-fluorouracil, capecitabine, vincristin, vinblastin and methotrexate, topoisomerase inhibitors (e.g., irinotecan, topotecan, camptothecin, etoposide) or any derivative related agent of the foregoing. Many of the above agents are also referred to as hormone therapy agents such as, for example, androgen receptor binding agents, gonadotropin-releasing hormone agonists and antagonists, androgen synthesis inhibitors, estrogen receptor binding agents as well as aromatase inhibitors.

Radiotherapies for first-line and subsequent therapy include factors that cause DNA damage and include what are commonly known as γ-rays, X-rays, and/or the directed delivery of radioisotopes to tumor cells. Other forms of DNA damaging factors include microwaves and UV-irradiation. It is likely that all of these factors affect a broad range of damage on DNA, on the precursors of DNA, on the replication and repair of DNA, and on the assembly and maintenance of chromosomes. Dosage ranges for X-rays may range from daily doses of 50 to 200 roentgens for prolonged periods of time (e.g., 3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

Immunotherapies generally rely on the use of immune effector cells and molecules to target and destroy cancer cells. The immune effector may be, for example, a tumor antigen or an antibody specific for some marker on the surface of a tumor cell. The tumor antigen or antibody alone may serve as an effector of therapy or it may recruit other cells to actually effect cell killing. An antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. Alternatively, a tumor antigen may stimulate a subject's immune system to target the specific tumor cells using cytotoxic T cells and NK cells. Immunotherapies include Sipuleucel-T (Provenge®) and the like.

A gene therapy includes a therapeutic polynucleotide is administered before, after, or at the same time as a combination therapy. Therapeutic genes may include an antisense version of an inducer of cellular proliferation (oncogene), an inhibitor of cellular proliferation (tumor suppressor), or an inducer of programmed cell death (pro-apoptotic gene).

Surgery of some type is performed for resectable castration resistant prostate cancers. Surgery types include preventative, diagnostic or staging, curative and palliative surgery and can be performed as a first-line and subsequent therapy. Surgery also includes prostatectomy and orchiectomy procedures.

In some embodiments, the wortmannin analogs described herein are administered as a second-line therapy after a first-line therapy becomes ineffective or the castration resistant prostate cancer is recurrent. In other embodiments, the wortmannin analogs described herein administered as a third-line therapy after the first- and second-line therapy fails. In further embodiments, individuals are preselected for having completed a first- or second-line therapy. In some instances, the wortmannin analogs described herein are administered to patients for whom prior androgen ablation therapy has failed. In other instances, the wortmannin analogs described herein are concurrently administered to patients undergoing androgen ablation therapy. In yet further instances, the wortmannin analogs described herein are administered to patients where the prostate cancer is hormone refractory or castration resistant.

In other embodiments, the wortmannin analogs described herein are administered to subjects who have undergone a surgery. In certain instances, the wortmannin analogs described herein are administered to subjects who had prostatectomy. In other instances, the wortmannin analogs described herein are administered to subjects who had orchiectomy.

In some embodiments of any of the methods described above, subjects, in some instances, are prescreened or preselected prior to treatment with a wortmannin analog to increase effectiveness of treatment. In some embodiments, subjects are preselected as to not having prior anti-cancer therapy with a PI-3 kinase inhibitor. In other embodiments, subjects are preselected as to not having other malignancies. Other malignancies, include but are not limited, to malignancies from other cancers. In yet other embodiments, subjects are preselected as to not having uncontrolled diabetes mellitus. In further embodiments, subjects are preselected as to not being positive for human immunodeficiency virus (HIV).

In some embodiments of any of the methods described above, subjects, in some instances, can also be prescreened or preselected for sensitivity and/or effectiveness of the wortmannin analogs described herein. A subject can be examined for certain biomarkers that allow the subject to be amenable to a wortmannin analog. For example, biomarkers such as phosphatase and tensin homolog (PTEN) mutations and activating mutations of PI-3K catalytic subunits may increase sensitivity to the wortmannin analogs described herein whereas other mutations such as Ras pathway mutations may decrease sensitivity. In some embodiments, a subject is preselected based on, for example, PTEN mutational status, PTEN copy number, PI3K gene amplification, EGFR activity, PI3K catalytic subunit alpha (PIK3CA) mutational status, K-ras mutational status, and/or B-raf mutational status. Additional biomarker candidates are contemplated in the subsequent sections.

In some embodiments of any of the methods described above, the wortmannin analog (e.g., a compound of Formula IA, Formula IB, Formula IIA, Formula IIB, PX-866 or PX-867) and/or a metabolite thereof is administered orally. In some embodiments of any of the methods described above, the wortmannin analog (e.g., a compound of Formula IA, Formula IB, Formula IIA, Formula IIB, PX-866 or PX-867) and/or a metabolite thereof is administered orally in the fasted state. In some embodiments of any of the methods described above, the wortmannin analog (e.g., a compound of Formula IA, Formula IB, Formula IIA, Formula IIB, PX-866 or PX-867) and/or a metabolite thereof is administered orally in the fed state.

### Pharmaceutical Compositions of Wortmannin Analogs

Pharmaceutical compositions containing wortmannin analogs can be administered in therapeutically effective amounts as pharmaceutical compositions by any conventional form and route known in the art including, but not limited to: injection, transdermal, nasal, pulmonary, vaginal, rectal, buccal, ocular, otic, local, topical, or oral administration. In certain embodiments, an injectable pharmaceutical composition of a wortmannin analog is an intramuscular, intravenous, subcutaneous, intranodal, intratumoral, intracisternal, intraperitoneal, or intradermal injection. In addition, the pharmaceutical composition containing wortmannin analogs may be provided in the form of a rapid release formulation, in the form of an extended release formulation, or in the form of an intermediate release formulation.

For oral administration, wortmannin analogs can be formulated readily by combining the active compounds with pharmaceutically acceptable carriers or excipients well known in the art. Such carriers enable the compounds described herein to be formulated as tablets, powders, pills, dragees, capsules, liquids, gels, syrups, elixirs, slurries, suspensions and the like, for oral ingestion by a patient to be treated.

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross linked croscarmellose sodium, polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push fit capsules made of gelatin, as well as soft, sealed capsules made of gelatin and a plasticizer, such as glycerol or sorbitol. The push fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration. In some embodiments, a wortmannin analog is in powder form and is directly filled into hard gelatin capsules.

For buccal or sublingual administration, the compositions may take the form of tablets, lozenges, or gels formulated in conventional manner.

Injectable compositions may involve for bolus injection or continuous infusion. An injectable composition of wortmannin analogs may be in a form suitable for parenteral or any other type of injection as a sterile suspensions, solutions or emulsions in oily or aqueous vehicles, and may contain formulatory agents such as suspending, stabilizing and/or dispersing agents. The composition may be formulated for intramuscular, intravenous, subcutaneous, intranodal, intratumoral, intracisternal, intraperitoneal, and/or intradermal injection. Pharmaceutical formulations for injection administration include aqueous solutions of the active compounds in water soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

In various embodiments, wortmannin analog compositions are in liquid form for ocular or otic delivery. Liquid forms include, by way of non-limiting example, neat liquids, solutions, suspensions, dispersions, colloids, foams and the like and can be formulated by known methods.

Wortmannin analogs can be administered topically and can be formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

Formulations suitable for transdermal administration of wortmannin analogs may employ transdermal delivery devices and transdermal delivery patches and can be lipophilic emulsions or buffered, aqueous solutions, dissolved and/or dispersed in a polymer or an adhesive. Such patches may be constructed for continuous, pulsatile, or on demand delivery of pharmaceutical agents. Still further, transdermal delivery of the wortmannin analogs can be accomplished by means of iontophoretic patches and the like. Additionally, transdermal patches can provide controlled delivery of the wortmannin analogs. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

For administration by inhalation for pulmonary or nasal delivery, wortmannin analogs maybe in a form as an aerosol, a mist or a powder. Pharmaceutical compositions of wortmannin analogs are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, such as, by way of example only, gelatin for use in an inhaler or insufflator may be formulated containing a powder mix of the compound and a suitable powder base such as lactose or starch.

Wortmannin analogs may also be formulated in rectal or vaginal compositions such as enemas, douches, gels, foams, aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

One may administer wortmannin analogs in a local rather than systemic manner, for example, via injection of the compound directly into an organ, often in a depot or sustained release formulation. Furthermore, one may administer pharmaceutical composition containing wortmannin analogs in a targeted drug delivery system, for example, in a liposome coated with organ-specific antibody. The liposomes will be targeted to and taken up selectively by the organ. Pharmaceutical compositions of wortmannin analogs may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. Pharmaceutical compositions comprising a wortmannin analogs may be manufactured in a conventional manner, such as, by way of example only, by means of conventional mixing, dissolving, granulating, dragee-making, levigating, emulsifying, encapsulating, entrapping or compression processes.

The pharmaceutical compositions will include at least one pharmaceutically acceptable carrier, diluent or excipient and a wortmannin analog described herein as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. In some situations, wortmannin analogs may exist as tautomers. All tautomers are included within the scope of the compounds presented herein. Additionally, wortmannin analogs described herein can exist in unsolvated as well as solvated forms with pharmaceutically acceptable solvents such as water, ethanol, and the like. The solvated forms of wortmannin analogs presented herein are also considered to be disclosed herein. In addition, the pharmaceutical compositions may include other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions can also contain other therapeutically valuable substances.

Methods for the preparation of compositions comprising wortmannin analogs described herein include formulating the wortmannin analogs with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The compositions may be in liquid solutions or suspensions, solid forms suitable for solution or suspension in a liquid prior to use, or as emulsions. These compositions may also contain minor amounts of nontoxic, auxiliary substances, such as wetting or emulsifying agents, pH buffering agents, and so forth.

Further forms of pharmaceutical compositions of wortmannin analogs can be integrated with other active agents, e.g., docetaxel, in a unitary dosage form for combination therapies. The unitary dosage forms can be formulated to release where both agents are released simultaneously or where there is sequential release of each agent via known modified release mechanisms including but not limited to timed release, delayed release, pH release, pulsatile release and the like.

In certain embodiments, pharmaceutical compositions of wortmannin analogs described herein are in unit dosage forms suitable for single administration of precise dosages. In unit dosage form, the formulation is divided into unit doses containing appropriate quantities of one or more compound. In specific embodiments, the unit dosage is in the form of a package containing discrete quantities of the formulation. Non-limiting examples are packaged tablets or capsules, and powders in vials or ampoules. Aqueous suspension compositions are optionally packaged in single-dose non-re-closeable containers. Alternatively, multiple-dose re-closeable containers are used, in which case it is typical to include a preservative in the composition. By way of example only, formulations for parenteral injection are, in some embodiments, presented in unit dosage form, which include, but are not limited to ampoules, or in multi-dose containers, with an added preservative.

A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety.

### Wortmannin Analogs Dosing

In one embodiment, a compound of Formula IA, IB, HA or IIB or any other PI-3 kinase inhibitor and/or wortmannin analog described herein is used in the preparation of medicaments for the treatment of cancers. In addition, a method for treating any of the diseases or conditions described herein in a subject in need of such treatment, involves administration of pharmaceutical compositions containing at least one compound of Formula IA, IB, IIA or IIB or any other PI-3 kinase inhibitor and/or wortmannin analog described herein, or a pharmaceutically acceptable salt, pharmaceutically active metabolite, pharmaceutically acceptable prodrug, pharmaceutically acceptable solvate thereof, or pharmaceutically acceptable polymorph in therapeutically effective amounts to said subject.

Dosages of wortmannin analogs described herein (e.g., compounds of Formula IA, IB, IIA or IIB or any other PI-3 kinase inhibitor and/or wortmannin analog described herein) can be determined by any suitable method. Maximum tolerated doses (MTD) and maximum response doses (MRD) can be determined via established animal and human experimental protocols as well as in the examples described herein. For example, toxicity and therapeutic efficacy of wortmannin analogs can be determined by standard pharmaceutical procedures in cell cultures or experimental animals, including, but not limited to, for determining the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Wortmannin analogs exhibiting high therapeutic indices are of interest. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized. Additional relative dosages, represented as a percent of maximal response or of maximum tolerated dose, are readily obtained via the protocols.

In some embodiments, the amount of a given wortmannin analog that corresponds to such an amount varies depending upon factors such as the particular compound, disease condition and its severity, the identity (e.g., weight, sex) of the subject or host in need of treatment, but can nevertheless be determined according to the particular circumstances surrounding the case, including, e.g., the specific agent being administered, the route of administration, the condition being treated, and the subject or host being treated.

In other embodiments, however, doses employed for adult human treatment are typically in the range of about 0.01 mg to about 5000 mg per day, or about 1 mg to about 1500 mg per day. In one embodiment, the desired dose is conveniently presented in a single dose or in divided doses administered simultaneously (or over a short period of time) or at appropriate intervals, for example as two, three, four or more sub-doses per day.

In some embodiments, wortmannin analogs are provided in a dose per day from about 0.01 mg to 1000 mg, from about 0.1 mg to about 100 mg, from about 1 to about 20, from about 2 mg to about 12 mg. In certain embodiments, wortmannin analogs are provided in a daily dose of about 0.01 mg, about 0.05 mg, about 0.1 mg, about 0.2 mg, about 0.4 mg, about 0.6 mg, about 0.8 mg, about 1 mg, about 1.5 mg, about 2 mg, about 2.5 mg, about 3 mg, about 4 mg, about 5 mg, about 6 mg, about 7 mg, about 8 mg, about 9 mg, about 10 mg, about 11 mg, about 12 mg, about 15 mg, about 20 mg, about 25 mg, about 30 mg, about 40 mg, about 50 mg, about 75 mg, about 100 mg, about 150 mg, about 200 mg, about 250 mg, about 300 mg, about 500, mg, about 750 mg, about 1000 mg, or more, or any range derivable therein. In certain instances, wortmannin analogs are provided in a dose per day of about 1 mg. In certain instances, wortmannin analogs are provided in a dose per day of about 2 mg. In certain instances, wortmannin analogs are provided in a dose per day of about 3 mg. In certain instances, wortmannin analogs are provided in a dose per day of about 4 mg. In certain instances, wortmannin analogs are provided in a dose per day of about 5 mg. In certain instances, wortmannin analogs are provided in a dose per day of about 6 mg. In certain instances, wortmannin analogs are provided in a dose per day of about 7 mg. In certain instances, wortmannin analogs are provided in a dose per day of about 8 mg. In certain instances, wortmannin analogs are provided in a dose per day of about 9 mg. In certain instances, wortmannin analogs are provided in a dose per day of about 10 mg. In certain instances, wortmannin analogs are provided in a dose per day of about 11 mg. In certain instances, wortmannin analogs are provided in a dose per day of about 12 mg. The dose per day described herein can be given once per day or multiple times per day in the form of sub-doses given b.i.d., t.i.d., q.i.d., or the like where the number of sub-doses equal the dose per day.

In further embodiments, the daily dosages appropriate for the compound of Formula IA, IB, HA or IIB or any other PI-3 kinase inhibitor and/or wortmannin analog described herein are from about 0.001 to about 100 mg/kg per body weight. In one embodiment, the daily dosages appropriate for the compound of Formula IA, IB, IIA or IIB or any other PI-3 kinase inhibitor and/or wortmannin analog described herein are from about 0.01 to about 10 mg/kg per body weight. In some embodiments, an indicated daily dosage in a large mammal, including, but not limited to, humans, is in the range from about 0.02 mg to about 1000 mg, conveniently administered in divided doses, including, but not limited to, up to four times a day. In one embodiment, the daily dosage is administered in extended release form. In certain embodiments, suitable unit dosage forms for oral administration comprise from about 1 to 500 mg active ingredient. In other embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

In other embodiments wortmannin analogs are provided at the maximum tolerated dose (MTD). In other embodiments, the amount of wortmannin analogs administered is from about 10% to about 90% of the maximum tolerated dose (MTD), from about 25% to about 75% of the MTD, or about 50% of the MTD. In particular embodiments, the amount of wortmannin analogs administered is from about 5%, 10%, 15%, 20%, 25%, 30%, 35%, 40%, 45%, 50%, 55%, 60%, 65%, 70%, 75%, 80%, 85%, 90%, 95%, 99%, or higher, or any range derivable therein, of the MTD.

### Administration of a Wortmannin Analog

Administration of a wortmannin analog is at dosages and compositions described herein or at other dose levels and compositions determined and contemplated by a medical practitioner.

In certain embodiments, the wortmannin analogs described herein are administered for prophylactic and/or therapeutic treatments. In certain therapeutic applications, the wortmannin analogs are administered to a patient already suffering from a cancer, in an amount sufficient to cure or at least partially arrest the symptoms of the cancer. Amounts effective for this use depend on the severity and course of the cancer, previous therapy, the patient's health status, weight, and response to the drugs, and the judgment of the treating physician. Therapeutically effective amounts are optionally determined by methods including, but not limited to, a dose escalation clinical trial, such as described in Example 1.

In prophylactic applications, wortmannin analogs described herein are administered to a patient susceptible to or otherwise at risk of a particular cancer. Such an amount is defined to be a “prophylactically effective amount or dose.” In this use, the precise amounts also depend on the patient's state of health, weight, and the like. When used in a patient, effective amounts for this use will depend on the severity and course of the cancer, previous therapy, the patient's health status and response to the drugs, and the judgment of the treating physician.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's cancer. In other embodiments, administration of a wortmannin analog continues until complete or partial response of a cancer.

In certain embodiments wherein a patient's status does improve, the dose of a wortmannin analog being administered may be temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

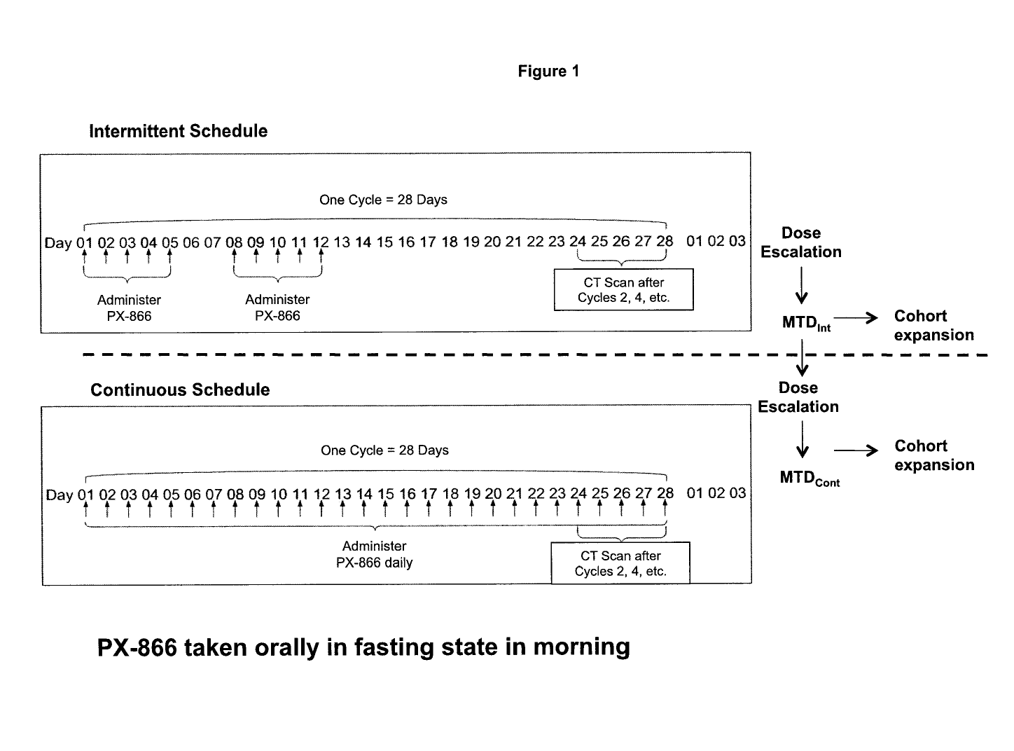

In some embodiments, compounds of Formula IA, IB, IIA or IIB or any other PI-3 kinase inhibitor and/or wortmannin analog described herein are administered chronically. For example, in some embodiments, a wortmannin analog (e.g., PX-866 and/or metabolite thereof) is administered as a continuous dose, i.e., administered daily to a subject. In some embodiments, a desired chronic dose is a low dose (e.g., between about 0.02 mg to about 20 mg per day) that is administered as a continuous dose as described herein in FIGS. 1-7 and in Example 1.

In some embodiments, compounds of Formula IA, IB, IIA or IIB or any other PI-3 kinase inhibitor and/or wortmannin analog described herein are administered intermittently (e.g. drug holiday that includes a period of time in which the compound is not administered or is administered in a reduced amount). In some embodiments, compounds of Formula Formula IA, IB, IIA or IIB or any other PI-3 kinase inhibitor and/or wortmannin analog described herein are administered in cycles that include: (a) a first period that includes daily administration of the compound of Formula IA, IB, IIA or IIB or any other PI-3 kinase inhibitor and/or wortmannin analog described herein; followed by (b) a second period that includes a dose reduction of the daily amount of the compound of Formula IA, IB, IIA or IIB or any other PI-3 kinase inhibitor and/or wortmannin analog described herein that is administered. In some embodiments, the compound of Formula IA, IB, IIA or IIB or any other PI-3 kinase inhibitor and/or wortmannin analog described herein is not administered in the second period. In some embodiments, the duration of the first and second periods, as well as the dose amounts of a PI-3 kinase inhibitor (for example, PX-866) are described herein. In some instances, a drug holiday or a dose reduction period is appropriate depending on the pharmacodynamic profile of the active agent.

Administration of a wortmannin analog (e.g., a compound of Formula IA, IB, IIA or IIB or any other PI-3 kinase inhibitor and/or wortmannin analog described herein) is, in some embodiments, provided daily to a subject. In other embodiments, wortmannin analogs (e.g., a compound of Formula IA, IB, IIA or IIB or any other PI-3 kinase inhibitor and/or wortmannin analog described herein) are administered every other day, every 2 days, every 3 days, every 4 days, every 5 days, every 6 days or every 7 days to a subject. In some embodiments, wortmannin analog (e.g., a compound of Formula IA, IB, IIA or IIB or any other PI-3 kinase inhibitor and/or wortmannin analog described herein) is administered as continuous dosing (e.g., daily dosing in a 28 day chemotherapy cycle).

Administration of a wortmannin analog can, in other embodiments, also be provided in an intermittent dosing schedule. Intermittent dosing schedules include administering a wortmannin analog for a number of days, withholding administration for a certain period of time, subsequently administering a wortmannin analog again with another subsequent withholding. In a non-limiting example, for a 28-day treatment cycle, a wortmannin analog can be administered for days 1-5 and 8-12. Other intermittent dosing schedules are contemplated that include administration of a wortmannin analog daily for one, two, three, four, five, six, seven, eight, nine or ten days, a withholding period of one, two, three, four, five, six, seven, eight, nine or ten days and an optional daily and withholding period similar or different from the previous administration within a treatment cycle.

In some embodiments, administration of a wortmannin analog is over a period of time of at least about 3 weeks, at least about 6 weeks, at least about 8 weeks, at least about 12 weeks, at least about 16 weeks, at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 68 weeks, at least about 72 weeks, at least about 90 weeks, at least about 100 weeks, at least about 110 weeks, and at least about 120 weeks. In certain instances, administration of a wortmannin analog is over 8 weeks. In other embodiments, administration of a wortmannin analog continues until complete or partial response.

Administration periods can be further defined as treatment cycles where a given number of days or weeks equates one treatment cycle. In some embodiments, one treatment cycle is an administration period of about 1 week, about 2 weeks, about 4 weeks, about 6 weeks, about 8 weeks, about 10 weeks, about 12 weeks or about 16 weeks. In certain embodiments, one treatment cycle is 8 weeks. Treatment cycles for administration of wortmannin analogs also include, but are not limited to 1 cycle, 2 cycles, 3 cycles, 4 cycles, 5 cycles, 6 cycles, 7 cycles, 8 cycles, 9 cycles, 10 cycles, 11 cycles, 12 cycles, 13 cycles, 14 cycles, 15 cycles, 16 cycles, 17 cycles, 18 cycles, 19 cycles, 20 cycles, 25 cycles, 30 cycles, 40 cycles, or more.

Dosages for wortmannin analogs can, in some embodiments, be the same for each treatment cycle or the dosages may vary per cycle. In some embodiments, a higher initial dose of a wortmannin analog is administered for the first cycle and a lower dose is administered for all subsequent cycles. In other embodiments, the wortmannin analog dosages are decreased gradually per administration for each cycle. In yet other embodiments, the wortmannin analog dosages are increased gradually per administration for each cycle.

In some embodiments, a wortmannin analog administration is withheld or given a “drug holiday” in one or more treatment cycles. For example, a wortmannin analog is administered for one treatment cycle and subsequently withheld for the next treatment cycle. In other embodiments, a wortmannin analog is withheld from a subject every other treatment cycle, every two treatment cycles, every three treatment cycles, every four treatment cycles, or every five treatment cycles.

In some embodiments when a wortmannin analog is administered orally, the oral administration is given to a subject who is in a fasted state. A fasted state refers to a subject who has gone without food or fasted for a certain period of time. General fasting periods include at least 4 hours, at least 6 hours, at least 8 hours, at least 10 hours, at least 12 hours, at least 14 hours and at least 16 hours without food. In some embodiments, a wortmannin analog is administered orally to a subject who is in a fasted state for at least 8 hours. In other embodiments, a wortmannin analog is administered orally to a subject who is in a fasted state for at least 10 hours. In yet other embodiments, a wortmannin analog is administered orally to a subject who is in a fasted state for at least 12 hours. In other embodiments, a wortmannin analog is administered orally to a subject who has fasted overnight.

In other embodiments when a wortmannin analog is administered orally, the oral administration is given to a subject who is in a fed state. A fed state refers to a subject who has taken food or has had a meal. In certain embodiments, a wortmannin analog is administered orally to a subject in a fed state 5 minutes post-meal, 10 minutes post-meal, 15 minutes post-meal, 20 minutes post-meal, 30 minutes post-meal, 40 minutes post-meal, 50 minutes post-meal, 1 hour post-meal, or 2 hours post-meal. In certain instances, a wortmannin analog is administered orally to a subject in a fed state 30 minutes post-meal. In other instances, a wortmannin analog is administered orally to a subject in a fed state 1 hour post-meal. In yet further embodiments, a wortmannin analog is administered orally to a subject with food.

In further embodiments described herein, the wortmannin analog is administered at a certain time of day for the entire administration period. For example, a wortmannin analog can be administered at a certain time in the morning, in the evening, or prior to bed. In certain instances, a wortmannin analog is administered in the morning. In other embodiments, a wortmannin analog can be administered at different times of the day for the entire administration period. For example, a wortmannin analog can be administered in 8:00 am in the morning for the first day, 12 pm noon for the next day or administration, 4 pm in the afternoon for the third day or administration, and so on.

Any administration of the wortmannin analogs described herein can be adjusted and modified accordingly via factoring conditions as a subject's response, age, sex, disease, etc at the beginning of treatment and throughout the course of the administration.

In any of the aforementioned embodiments, the wortmannin analog is PX-866, or salt, solvate, or polymorph thereof. In any of the aforementioned embodiments, the wortmannin analog is 17-hydroxy PX-866, or salt, solvate, or polymorph thereof.

### Further Combinations

The treatment of a cancer (e.g., glioblastoma or castration resistant prostate cancer) in a subject with a wortmannin analog described herein encompass additional therapies and treatment regimens with other agents in some embodiments. Such additional therapies and treatment regimens can include another anti-cancer therapy in some embodiments. Alternatively, in other embodiments, additional therapies and treatment regimens include other agents used to treat adjunct conditions associated with the cancer or a side effect from the wortmannin analog in the therapy. In further embodiments, adjuvants or enhancers are administered with a wortmannin analog described herein.

Additional anti-cancer therapies include chemotherapy, radiotherapy, immunotherapy, gene therapy, surgery or other therapies that are capable of negatively affecting cancer in a patient, such as for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

Chemotherapies for combinations with a wortmannin analog include, but are not limited to, hormone modulators, androgen receptor binding agents (e.g., anti-androgens, bicalutamide, flutamide, nilutamide, MDV3100), gonadotropin-releasing hormone agonists and antagonists (e.g., leuprolide, buserelin, histrelin, goserelin, deslorelin, nafarelin, abarelix, cetrorelix, ganirelix degarelix), androgen synthesis inhibitors (abiraterone, TOK-001), temozolomide, mitozolomide, dacarbazine, cisplatin (CDDP), carboplatin, procarbazine, mechlorethamine, cyclophosphamide, camptothecin, ifosfamide, melphalan, chlorambucil, busulfan, nitrosurea, dactinomycin, anthracyclines (e.g., daunorubicin, doxorubicin, epirubicin, idarubicin), bleomycin, plicomycin, mitomycin, etoposide (VP16), tamoxifen, raloxifene, estrogen receptor binding agents, docetaxel, paclitaxel, cabazitaxel, gemcitabine, navelbine, farnesyl-protein transferase inhibitors, transplatinum, 5-fluorouracil, capecitabine, vincristin, vinblastin and methotrexate, topoisomerase inhibitors (e.g., irinotecan, topotecan, camptothecin, etoposide) or any derivative related agent of the foregoing. Many of the above agents are also referred to as hormone therapy agents such as, for example, androgen receptor binding agents, gonadotropin-releasing hormone agonists and antagonists, androgen synthesis inhibitors, estrogen receptor binding agents as well as aromatase inhibitors.

In some embodiments, the wortmannin analogs provided herein are administered with a chemotherapy or hormone therapy. In certain embodiments, the wortmannin analogs provided herein are administered with an anti-androgen. In other embodiments, the wortmannin analogs provided herein are administered with a gonadotropin-releasing hormone agonist or antagonist. In further embodiments, the wortmannin analogs provided herein are administered with an androgen synthesis inhibitor.

In some embodiments, the wortmannin analogs provided herein are administered with a DNA alkylating agent. In other embodiments, the wortmannin analogs provided herein are administered with temozolomide. In yet other embodiments, the wortmannin analogs provided herein are administered with topotecan. In further embodiments, the wortmannin analogs provided herein are administered with docetaxel.

Radiotherapies include factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is likely that all of these factors affect a broad range of damage on DNA, on the precursors of DNA, on the replication and repair of DNA, and on the assembly and maintenance of chromosomes. Dosage ranges for X-rays may range from daily doses of 50 to 200 roentgens for prolonged periods of time (e.g., 3 to 4 weeks), to single doses of 2000 to 6000 roentgens. Dosage ranges for radioisotopes vary widely, and depend on the half-life of the isotope, the strength and type of radiation emitted, and the uptake by the neoplastic cells. In some embodiments, the wortmannin analogs described herein are administered with a radiotherapy.

Immunotherapies, generally, rely on the use of immune effector cells and molecules to target and destroy cancer cells. The immune effector may be, for example, a tumor antigen or an antibody specific for some marker on the surface of a tumor cell. The tumor antigen or antibody alone may serve as an effector of therapy or it may recruit other cells to actually effect cell killing. An antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. Alternatively, an tumor antigen may stimulate a subject's immune system to target the specific tumor cells using cytotoxic T cells and NK cells. In some embodiments, the wortmannin analogs described herein are administered with an immunotherapy. In some embodiments, the wortmannin analogs described herein are administered with Sipuleucel-T (Provenge®). Exemplary immunotherapies for glioblastoma include bevacizumab, a monoclonal antibody targeting the vascular endothelial growth factor receptor (VEGF-R). In other embodiments, the wortmannin analogs described herein are administered with bevacizumab. In yet further embodiments, the wortmannin analogs provided herein are administered with cetuximab.

In other embodiments, an additional anti-cancer therapy is a gene therapy in which a therapeutic polynucleotide is administered before, after, or at the same time as a combination therapy. Therapeutic genes may include an antisense version of an inducer of cellular proliferation (oncogene), an inhibitor of cellular proliferation (tumor suppressor), or an inducer of programmed cell death (pro-apoptotic gene). In some embodiments, the wortmannin analogs described herein are administered with a gene therapy.

In further embodiments, surgery of some type is performed in conjunction with the wortmannin analogs described herein. Surgery types include preventative, diagnostic or staging, curative and palliative surgery and can be performed prior to, during, or subsequent to the wortmannin analog therapy.

The mammalian target of rapamycin (mTOR) is a highly conserved intracellular serine/threonine kinase and a major downstream component in the PI3K pathway. Certain studies demonstrate that the PI3K-Akt-mTOR pathway mediates the response induced by EGFR activation. Accordingly, in some embodiments, methods of treatment of cancer described herein comprise administration of small molecule EGFR tyrosine kinase inhibitors (e.g., gefitinib, erlotinib or the like) in combination with wortmannin analogs for prevention, delayed progression, reversal and/or partial reversal of established cancers and/or cancers that are refractory to other treatments. In some embodiments, methods of treatment of cancer described herein comprise administration of wortmannin analogs in combination with small molecule mTor inhibitors including and not limited to rapamycin, temsirolimus, deforolimus, everolimus, BEZ235 or the like.

In some embodiments, an additional agent used to treat adjunct conditions associated with the cancer (e.g., glioblastoma or castration resistant prostate cancer) or a side effect from the wortmannin analog in the treatment. Additional agents include, but are not limited to, anti-inflammatories, anti-emetics, anti-diarrheals and analgesics. In certain instances, the additional agents are administered prophylactically or as a pre-treatment prior to the wortmannin analog. In other instances, the additional agents are administered on a needed basis, i.e., when a condition or side effect arises.

Anti-inflammatories can be used to treat or reduce the incidence and severity of, for example, inflammatory conditions, fluid retention or hypersensitivity reactions that result from the wortmannin analog and/or conditions from the cancer (e.g., glioblastoma or castration resistant prostate cancer). Anti-inflammatories are often given to patients with glioblastoma to reduce peritumoral edema, diminish mass effect, lower intracranial pressure and reduce headache or drowsiness. Anti-inflammatories include, but are not limited to corticosteroids (e.g., dexamethasone, prednisone, hydrocortisone, betamethasone, triamcinolone and the like); NSAIDS such as arylcarboxylic acids (salicylic acid, acetylsalicylic acid, diflunisal, choline magnesium trisalicylate, salicylate, benorylate, flufenamic acid, mefenamic acid, meclofenamic acid and triflumic acid), arylalkanoic acids (diclofenac, fenclofenac, alclofenac, fentiazac, ibuprofen, flurbiprofen, ketoprofen, naproxen, fenoprofen, fenbufen, suprofen, indoprofen, tiaprofenic acid, benoxaprofen, pirprofen, tolmetin, zomepirac, clopinac, indomethacin and sulindac) and enolic acids (phenylbutazone, oxyphenbutazone, azapropazone, feprazone, piroxicam, and isoxicam); and anti-histamines such as cimetidine, ranitidine, famotidine and nizatidine.

Anti-emetics can be used to treat nausea or vomiting associated with the cancer (e.g., glioblastoma or castration resistant prostate cancer) or administration of the wortmannin analog. Anti-emetics include 5-HT receptor antagonists (ondansetron, granisetron, dolasetron, tropisetron, palonosetron, mirtazapine, etc.), dopamine antagonists (haloperidol, droperidol, prochlorperazine, etc.), antihistamines such as H₁ antagonists, (promethazine, diphenhydramine, meclizine, etc.), benzodiazepines (lorazepam, midazolam), cannabinoids, and dexamethasone. Other known anti-emetics can be used as in conjunction with the wortmannin analog in some embodiments.

Anti-diarrheals can be used to treat or prevent diarrhea associated with the cancer (e.g., glioblastoma or castration resistant prostate cancer) or administration of the wortmannin analog. Anti-diarrheals include bismuth subsalicylate, loperamide, diphenoxylate, difenoxin, as well as other opioids.

Analgesics can be used to acute or chronic pain associated with the cancer (e.g., glioblastoma or castration resistant prostate cancer) or administration of the wortmannin analog. Analgesics include acetaminophen, NSAIDS and opioid drugs (morphine, hydromorphone, fentanyl, tramadol, oxymorphone, oxycodone, hydrocodone, etc.) and COX-2 inhibitors.

In further embodiments; other additional agents for use with wortmannin analogs described herein include immunosuppressants such as, for example, corticosteroids, gamma-interferon, Serum Amyloid P, azathioprine, penicillamine, cyclosporine, mycophenolate mofetil, or the like. Other additional therapeutic agents include colchicine, perfenidone or the like.

### Effects of Treatment

Treatment with a wortmannin analog described herein may result in various effects. One effect of treating a subject having cancer (e.g., glioblastoma, castration resistant prostate cancer or the like) with a wortmannin analog described herein is an increase in the length of survival. Similarly, administering a described wortmannin analog to a subject may impact that subject's “quality of life” or “health-related quality of life.” Moreover, in certain subjects, treatment with a wortmannin analog described herein results in modulating assessed biomarkers including, but not limited to, decreases in phosphatase and tensin homolog (PTEN) mutational status, PI3K gene amplification, PI3K catalytic subunit alpha (PIK3CA) mutational status, EGFR mutational status, K-ras mutational status, AKT phosphorylation status, androgen receptor copy number and/or B-raf mutational status as well as biomarkers specific in various cancers.

Comparisons of the effects of treatment with a wortmannin analog described herein can be made between treated subjects and subjects who are either undergoing no care, subjects who are undergoing a standard of care (SOC) or subjects who receive different wortmannin analog described herein. SOC comprises many alternative types of care that do not include treatment with a wortmannin analog described herein. For example, SOC, although usually discretionary depending on the circumstances, may include psychosocial support, analgesics, and nutritional support. In some embodiments, comparison of the effects of treatment will be made between subjects receiving differing amounts of a wortmannin analog described herein. In yet further embodiments, individuals will undergo SOC in conjunction with treatment with a wortmannin analog described herein.

In some embodiments, before treatment of a subject having cancer (e.g., glioblastoma, castration resistant prostate cancer or the like) with a wortmannin analog described herein, the subject may undergo pre-treatment evaluation. A non-limiting example of a pre-treatment evaluation includes a complete history and physical examination. The physical examination may include such things as a CT scan, MRI brain scan, X-ray, PET scan or bone scan. Pre-treatment evaluation may also include neurological exams, hematology (CBC, differential, platelets) and biochemistry (serum creatine, bilirubin, aminotransferase AST and ALT, total protein, fasting glucose, etc.) assessment. Subjects may also undergo treatment evaluations during the course of treatment. A treatment evaluation may include monitoring a subject's vital signs, inspecting injection sites if the wortmannin analog is administered via injection, and analyzing blood samples.

In some embodiments, a treated subject with a described wortmannin analog, may have treatment effects evaluated by determining: a) tumor size, (b) tumor location, (c) nodal stage, (d) growth rate of the cancer, (e) survival rate of the subject, (f) changes in the subject's cancer symptoms, (g) changes in the subject's Prostate Specific Antigen (PSA) concentration, (h) changes in the subject's PSA concentration doubling rate, (i) changes in the subject's biomarkers, or (i) changes in the subject's quality of life.

In some other embodiments, a treated subject with glioblastoma with a described wortmannin analog, may have treatment effects evaluated by determining: (a) glioblastoma size, (b) glioblastoma location, (c) nodal stage, (d) growth rate of the glioblastoma, (e) survival rate of the subject, (f) changes in the subject's glioblastoma symptoms, (g) changes in the subject's biomarkers, or (h) changes in the subject's quality of life. Treatment effects can be determined by any standardized criteria including those described in MacDonald et al, J Clin Oncol. 1990; 8(7):1277-1280.

Survival rates can be determined by comparing the current number of survivors with the number of individuals who started treatment with a described wortmannin analog. In other embodiments, survival rates can be compared to published survival rates for a particular type of cancer. In yet other embodiments, survival rates can be compared to survival rates of individuals treated with different wortmannin analogs. In general, the survival rate may be measured at any time following the start of treatment.

For example, the survival rate may be measured at less than 6 months following the start of treatment, greater than 6 months but less than a year, a year or greater but less than 2 years, 2 years or greater but less than 5 years, or 5 or greater years. In some embodiments, an increased survival rate will be evidence that a described wortmannin analog has effects on a particular subject.

In some embodiments, an effect of treating a subject having cancer a wortmannin analog described herein is maintenance or an increase in a subject's quality of life. Clinicians and regulatory agencies recognize that a subject's “quality of life” (“QoL”) is an important endpoint in cancer clinical trials. See, for instance, Litwin et al., JAMA. 1995; 273(2): 129-135; Miller et al., Journal of Clin. One. 2005; 23(12): 2772-2780, Bunston et al., Neurosurgical Focus. 1998; 4(5):e7, Bampoe et al., Journal of Neurosurgery. 2000; 93(6):917-926 and Steinbach et al., Neurology. 2006; 66(2):239-242, which are each incorporated herein by reference.

Four important quality of life indicators are physical and occupational function, psychologic state, social interaction, and somatic sensations. For example, questionnaires akin to lung cancer questionnaires from the European Organization for Research and Treatment of Cancer (“EORTC”) and the Functional Assessment of Cancer Therapy (“FACT-L”), are used to assess specifically an individual's health-related quality of life before, during, and after treatment with a wortmannin analog described herein.

In various embodiments, the above evaluations may be used in conjunction with assessments according to various subscales that monitor a subject's Physical Well-being (PWB), Social/Family Well-being (SWB), Emotional Well-being (EWB), Functional Well-being (FWB), and, for example, a Castration Resistant Prostate Cancer Symptom subscale (CRPCBS) akin to the Lung Cancer Symptom subscale (LCS) from FACT-L/EORTC. Depending on which “Well-being” scores are combined, one may obtain a “FACT-L score” (the sum of all of the subscales) or a “Trial Outcome Score (TOI)” (the sum of the PWB, FWB, and CRPCBS subscales). The TOI is a reliable indicator of meaningful change in quality of life. See, Cella et al., J. Clin. Epidemiol., 55(3):285-95 (2002).

A subject may be assessed for their FACT-L and TOI scores before, during, and after treatment with a wortmannin analog described herein. For instance, the TOI score may be taken at baseline, i.e., pre-treatment, and then at various intervals after treatment has started, i.e., at 4 weeks, 8 weeks, 19 weeks, 31 weeks, or 43 weeks, or longer. These various intervals are examples only and the quality of life indicators may be taken at any appropriate time. For example, the first TOI score may be taken after the first treatment, instead of at a baseline. Then, the change in scores between various time points may be calculated to determine trends relating to improving, worsening, or maintaining of quality of life.

It has been calculated that a decrease of 3 points or more from baseline for an exemplary CRPCBS is a clinically meaningful worsening in castration resistant prostate cancer symptoms and an increase in 3 or more points is a clinically meaningful improvement in castration resistant prostate cancer symptoms. Likewise for TOI scores, a decrease of 7 or more points indicates a worsening in quality of life, while an increase of 7 or more points indicates an improvement in quality of life. Similar subscales can be developed for other cancers such as glioblastomas.

In some embodiments, a clinical improvement in cancer (e.g., glioblastoma, castration resistant prostate cancer or the like) symptoms or quality of life demonstrates that a described wortmannin analog has effects on a particular subject.

Administering a wortmannin analog described herein may be useful in improving or maintaining the quality of life of treated subjects that have castration resistant prostate cancer. In measuring the effect on the quality of life, an effect size can be determined from baseline or from any treatment point. In some embodiments, an effect size of between 0.2 to <0.49 indicates a small effect, 0.5 to 0.79 indicates a moderate effect, and 0.8 or greater indicates a large effect for the above TOI score. These numbers are examples only and the effect size may change with treatment of certain subjects.

Administration of a wortmannin analog described herein may also be useful in preventing the worsening in quality of life seen over time in many cancer patients. For example, in some embodiments, administration of a wortmannin analog described herein may result in quality of life indexes that essentially remain unchanged or do not reach the level of worsening or improving quality of life.

In other embodiments, the present treatments described herein encompasses improving or maintaining the quality of life or improving or cancer (e.g., glioblastoma, castration resistant prostate cancer or the like) symptoms in an individual diagnosed with castration resistant prostate cancer by determining the individual's TOI or specific cancer subscale scores before, during, and after treatment with a wortmannin analog described herein.

In other embodiments, the response of subjects to a wortmannin analog described herein is measured by changes in certain biomarkers including, but not limited, decreases in phosphatase and tensin homolog (PTEN) mutational status, PI3K gene amplification, PI3K catalytic subunit alpha (PIK3CA) mutational status, K-ras mutational status, AKT phosphorylation status, androgen receptor copy number and/or B-raf mutational status. Biomarkers include other changes in copy number, nucleotide and protein concentrations, and/or mutational status in other genes involved in one of the PI-3K signal transduction pathways. The effects of a wortmannin analog on biomarkers can be measured at any time. For example, although a PTEN copy number can be compared to a baseline value, PTEN copy number may also be compared between treatment points or between a specific treatment point and the end of treatment.

In yet other embodiments, the response of subjects with prostate cancer to a wortmannin analog described herein is measured by changes in prostate specific antigen (“PSA”) concentrations, a stabilization of PSA concentrations, or a decrease in PSA doubling time. In individuals with prostate cancer, it is reported, for instance, that prostate-specific antigen (“PSA”) levels in the blood tend to rise when the prostate gland enlarges. Accordingly, PSA reportedly is a major biological or tumor marker for prostate cancer. In individuals with more advanced disease, treatment-induced decline in PSA correlates with improved survival (Scher, et al., J. Natl. Cancer Inst.; 91(3):244-51 (1999)).

In further embodiments, the response of subjects to a wortmannin analog described herein is measured using tests of immune function on a cancer. In some embodiments, the results from T-cell proliferation response assays will be used to determine whether a wortmannin analog described herein has an effect on a subject. Results from these assays may also be used to determine individual response to the formulations during different time points during the course of the treatment. Comparison of the T-cell proliferation response may be undertaken to compare pre-treatment versus post-treatment response as well as to compare immune responses within treatment.

### Kits/Articles of Manufacture

For use in the wortmannin analog treatments described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein including a wortmannin analog. Suitable containers include, for example, bottles, vials, syringes, and test tubes. The containers can be formed from a variety of materials such as glass or plastic.

A kit will typically may comprise one or more additional containers, each with one or more of various materials (such as reagents, optionally in concentrated form, and/or devices) desirable from a commercial and user standpoint for a wortmannin analog described herein. Non-limiting examples of such materials include, but not limited to, buffers, diluents, filters, needles, syringes; carrier, package, container, vial and/or tube labels listing contents and/or instructions for use, and package inserts with instructions for use associated with a wortmannin analog. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

Kits can be supplied and manufactured according to dosages or administration methods described herein. For example, a kit can be supplied with a container for a 1, 3, 5, or 10 treatment cycle of a wortmannin analog.

### DEFINITIONS

Unless defined otherwise, all technical and scientific terms used herein have the same meanings as commonly understood by one of ordinary skill in the art. Although any methods and materials similar or equivalent to those described herein can be used in the practice or testing of embodiments described herein, certain preferred methods, devices, and materials are now described.

As used herein and in the appended claims, the singular forms “a”, “an”, and “the” include plural reference unless the context clearly dictates otherwise. Thus, for example, reference to “a cell” is a reference to one or more cells and equivalents thereof known to those skilled in the art, and so forth.

The term “about” is used to indicate that a value includes the standard level of error for the device or method being employed to determine the value. The use of the term “or” in the claims is used to mean “and/or” unless explicitly indicated to refer to alternatives only or the alternatives are mutually exclusive, although the disclosure supports a definition that refers to only alternatives and to “and/or.” The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

“Optional” or “optionally” may be taken to mean that the subsequently described structure, event or circumstance may or may not occur, and that the description includes instances where the events occurs and instances where it does not.

“Administering” when used in conjunction with a therapeutic means to administer a therapeutic systemically or locally, as directly into or onto a target tissue, or to administer a therapeutic to a patient whereby the therapeutic positively impacts the tissue to which it is targeted. Thus, as used herein, the term “administering”, when used in conjunction with a wortmannin analog or metabolite thereof, can include, but is not limited to, providing a wortmannin analog or metabolite thereof into or onto the target tissue; providing a wortmannin analog or metabolite thereof systemically to a patient by, e.g., intravenous injection whereby the therapeutic reaches the target tissue or cells. “Administering” a composition may be accomplished by injection, topical administration, and oral administration or by other methods alone or in combination with other known techniques.

As used herein, the term “therapeutic” means an agent utilized to treat, combat, ameliorate, prevent or improve an unwanted condition or disease of a patient. In some embodiments, a therapeutic agent is directed to the treatment and/or the amelioration of, reversal of, or stabilization of the symptoms of a cancer described herein

The term “animal” as used herein includes, but is not limited to, humans and non-human vertebrates such as wild, domestic and farm animals. As used herein, the terms “patient,” “subject” and “individual” are intended to include living organisms in which certain conditions as described herein can occur. Examples include humans, monkeys, cows, sheep, goats, dogs, cats, mice, rats, and transgenic species thereof. In a preferred embodiment, the patient is a primate. In certain embodiments, the primate or subject is a human. Other examples of subjects include experimental animals such as mice, rats, dogs, cats, goats, sheep, pigs, and cows. The experimental animal can be an animal model for a disorder, e.g., a transgenic mouse with a glioblastoma pathology. A patient can be a human suffering from glioblastoma and variants or etiological forms.

The term “irreversible inhibitor” refers to an inhibitor that forms a covalent bond with the target moiety, in this case, PI-3 kinase.

By “pharmaceutically acceptable”, it is meant the carrier, diluent or excipient must be compatible with the other ingredients of the formulation and not deleterious to the recipient thereof.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

A “therapeutically effective amount” or “effective amount” as used herein refers to the amount of active compound or pharmaceutical agent that elicits a biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease, (2) inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) ameliorating the disease; for example, ameliorating a disease, condition or disorder in an individual that is experiencing or displaying the pathology or symptomatology of the disease, condition or disorder (i.e., reversing the pathology and/or symptomatology). As such, a non-limiting example of a “therapeutically effective amount” or “effective amount” of a composition of the present disclosure may be used to inhibit, block, or reverse the activation, migration, or proliferation of cells or to effectively treat cancer or ameliorate the symptoms of cancer.

The terms “treat,” “treated,” “treatment,” or “treating” as used herein refers to both therapeutic treatment in some embodiments and prophylactic or preventative measures in other embodiments, wherein the object is to prevent or slow (lessen) an undesired physiological condition, disorder or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. A prophylactic benefit of treatment includes prevention of a condition, retarding the progress of a condition, stabilization of a condition, or decreasing the likelihood of occurrence of a condition. As used herein, “treat,” “treated,” “treatment,” or “treating” includes prophylaxis in some embodiments.

As used herein, “continuous dosing” means administration of at least one dose of a compound (e.g., a PI-3 kinase inhibitor and/or metabolite thereof) daily for a period of at least 7 days. In some embodiments, continuous dosing means administration of at least one dose of a compound (e.g., a PI-3 kinase inhibitor and/or metabolite thereof) daily for 1 week. In some embodiments, continuous dosing means administration of at least one dose of a compound (e.g., a PI-3 kinase inhibitor and/or metabolite thereof) daily for 2 weeks. In some embodiments, continuous dosing means administration of at least one dose of a compound (e.g., a PI-3 kinase inhibitor and/or metabolite thereof) daily for 3 weeks. In some embodiments, continuous dosing means administration of at least one dose of a compound (e.g., a PI-3 kinase inhibitor and/or metabolite thereof) daily for 4 weeks. In some embodiments, continuous dosing means administration of at least one dose of a compound (e.g., a PI-3 kinase inhibitor and/or metabolite thereof) daily for 5 or more weeks.

Optionally, in some embodiments, continuous dosing alternates with a drug holiday in a cyclical treatment regimen. Accordingly, by way of example, in some embodiments, continuous dosing means administration of a first cycle of at least one dose of a compound (e.g., a PI-3 kinase inhibitor and/or metabolite thereof) daily for a period of at least one week followed by a drug holiday of up to two weeks, followed by administration of one or more further cycles of administration of at least one dose of a compound (e.g., a PI-3 kinase inhibitor and/or metabolite thereof) daily for a period of at least one week followed by a drug holiday of up to two weeks. In some embodiments, continuous dosing means administration of a first cycle of at least one dose of a compound (e.g., a PI-3 kinase inhibitor and/or metabolite thereof) daily for a period of at least 2 weeks followed by a drug holiday of up to two weeks, followed by administration of one or more further cycles of administration of at least one dose of a compound (e.g., a PI-3 kinase inhibitor and/or metabolite thereof) daily for a period of at least 2 weeks followed by a drug holiday of up to 2 weeks. In some embodiments, continuous dosing means administration of a first cycle of at least one dose of a compound (e.g., a PI-3 kinase inhibitor and/or metabolite thereof) daily for a period of at least 3 weeks followed by a drug holiday of up to two weeks, followed by administration of one or more further cycles of administration of at least one dose of a compound (e.g., a PI-3 kinase inhibitor and/or metabolite thereof) daily for a period of at least 3 weeks followed by a drug holiday of up to 2 weeks. In some embodiments, continuous dosing means administration of a first cycle of at least one dose of a compound (e.g., a PI-3 kinase inhibitor and/or metabolite thereof) daily for a period of at least 4 weeks followed by a drug holiday of up to two weeks, followed by administration of one or more further cycles of administration of at least one dose of a compound (e.g., a PI-3 kinase inhibitor and/or metabolite thereof) daily for a period of at least 4 weeks followed by a drug holiday of up to 2 weeks. In some embodiments, continuous dosing means administration of a first cycle of at least one dose of a compound (e.g., a PI-3 kinase inhibitor and/or metabolite thereof) daily for a period of 5 or more weeks followed by a drug holiday of up to two weeks, followed by administration of one or more further cycles of administration of at least one dose of a compound (e.g., a PI-3 kinase inhibitor and/or metabolite thereof) daily for a period of 5 or more weeks followed by a drug holiday of up to 2 weeks.

In some embodiments of a continuous dosing regimen, the drug holiday between two cycles of dosing is about 2 weeks. In some embodiments of a continuous dosing regimen, the drug holiday between two cycles of dosing is about 10 days. In some embodiments of a continuous dosing regimen, the drug holiday between two cycles of dosing is about 1 week. In some embodiments of a continuous dosing regimen, the drug holiday between two cycles of dosing is about 5 days. In some embodiments of a continuous dosing regimen, the drug holiday between two cycles of dosing is about 3 days.

As used herein, “intermittent dosing” means administration of a first cycle of at least one dose of a compound (e.g., a PI-3 kinase inhibitor and/or metabolite thereof) daily for a period of between about 2 to about 5 days, followed by a drug-free period of between about 2 to about 25 days, followed by one or more such cycles.

The term “wortmannin analog” or “analog of wortmannin” refers to any compounds in which one or more atoms, functional groups, or substructures in wortmannin have been replaced with different atoms, groups, or substructures while retaining or improving upon the functional activity of wortmannin and/or improving PK profiles and/or reducing toxicity of wortmannin.

### EXAMPLES

### Example 1

### A Phase I Trial of Oral PX-866 in Patients with Advanced Solid Tumors

This was an Open-label dose escalation study with expansion cohort at Maximal Tolerated Dose (MTD), 3+3 design and to test efficacy of 2 dosing schedules (intermittent and continuous). PX-866 was administered as an oral dose.

### Study Objectives

The primary and secondary objectives were tested using two different dosing regimens: 10 days of drug administration and daily administration for 28 days.

### Primary:

To determine the MTD of PX-866 when administered to patients with advanced metastatic cancers.
To evaluate the safety profile of PX-866 when administered orally on a 28 day schedule.
To evaluate pharmacodynamic measures of the effects of PX-866 on the phosphatidylinositol-3 kinase (PI-3K) pathway and related tumor markers.
To determine the PK profile of PX-866 when adminstered orally on a 28 day schedule.

### Secondary:

To evaluate the anti-tumor activity of PX-866 in patients with advanced malignancies.

### Selected Eligibility Criteria

Inclusion Criteria:
≧18 years at time of consent
Able to give an informed consent
Has a histologically or cytologically confirmed diagnosis of advanced solid tumor and has failed or is intolerant of standard therapy, or for whom standard therapy does not exist
Eastern Cooperative Oncology Group (ECOG) performance of 0 or 1
Life expectancy of at least 12 weeks
Discontinued prior chemotherapy or other investigational agents for at least three weeks prior to receiving the first dose of study drug (six weeks for mitomycin C, nitrosureas, vaccines, or antibody therapy) and recovered from the toxic effects of the prior treatment (recovered to baseline or ≦grade 1 per Common Toxicity Criteria for Adverse Events
Discontinued any radiation therapy for at least four weeks and have recovered from all radiation-related toxicities (recovered to baseline or ≦CTCAE grade 1) prior to receiving the first dose of study drug. Palliative radiation of 10 fractions or less is permitted and a four week interval is not necessary (also allowed during therapy).
Laboratory requirements:

| WBC count | >3,000 | cells/μL; |
|---|---|---|
| Platelets | >100,000/μL | |
| Hemoglobin | >9 | g/dL |
| ANC | >1,500 | cells/μL |
| Bilirubin | >1.5 | mg/dL |
| Aminotransferases (ALT and AST) | <2.5 × ULN or <5 × ULN due | |
| to metastatic disease | | |
| Serum Creatinine | <1.5 | mg/dL |

Women of childbearing potential agree to use adequate contraception (hormonal or barrier method; abstinence) prior to study entry and for the duration of study participation.

Exclusion Criteria:
Any active infection
Known diabetes or fasting blood glucose >160 mg/dL
Known HIV
Any serious concomitant systemic disorders that in the opinion of the investigator would place the patient at excessive or unacceptable risk of toxicity
Surgery within the four weeks prior to the first dose of PX-866
Significant central nervous system (CNS) or psychiatric disorder(s) that preclude the ability of the patient to provide informed consent
Known or suspected brain metastases that have not received adequate therapy
Patients with a history of seizures, non-healing wounds, or arterial thrombosis
Patients with unstable atrial or ventricular arrhythmias requiring control by medication
Patients who are breastfeeding or pregnant
Patients with total gastrectomy, partial bowel obstruction or any gastrointestinal condition that may interfere with absorption of the study medication
Any condition that could jeopardize the safety of the patient and compliance with the protocol

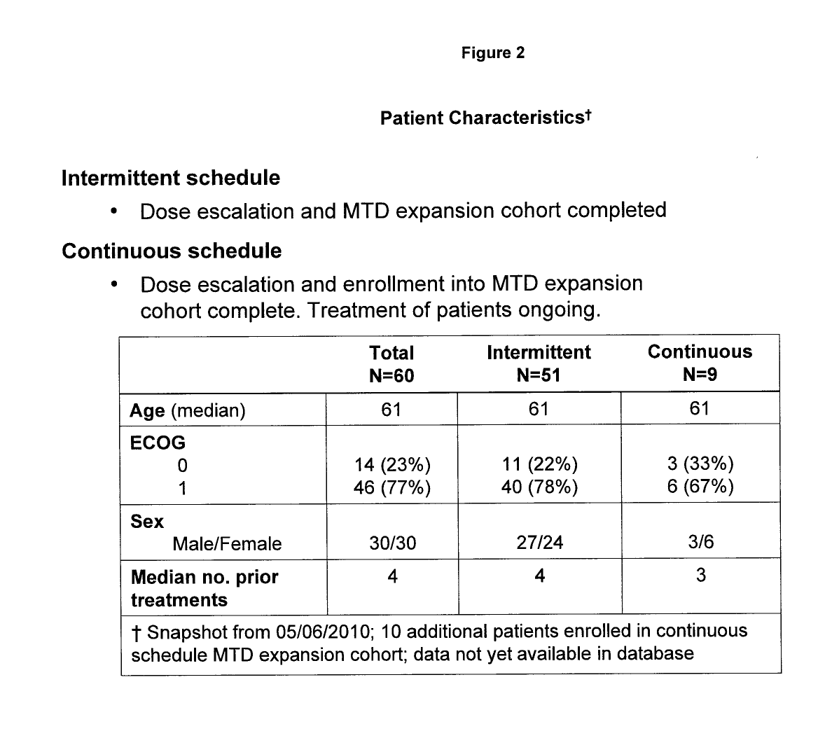

FIG. 2 describes a breakdown of patient characteristic from a May 6, 2010 snapshot.

Safety Levels with Intermittent Dosing

Intermittent schedule: 10 dose levels tested (0.5-16 mg). The starting dose level was 0.5 mg. Doses increased as follows: 100% escalation up to 2 mg, 50% escalation up to 4.5 mg, and approximately 30% escalation until the MTD is identified. The highest dose level at which no more than 1/6 patients experiences DLT was declared the MTD. The resulting dose levels were: 0.5, 1, 2, 3, 4.5, 6, 8, 10, 12 and 16 mg.

FIG. 1 illustrates the dosing schedule for intermittent dosing where PX-866 was given to patients on days 1-5 and 8-12 of a 28-day cycle.

At a dose of 16 mg per day, Dose limiting toxicity (DLT) was observed in 2/5 patients treated at 16 mg. Grade 3 diarrhea (n=1); Grade 3 AST (n=1) were observed.

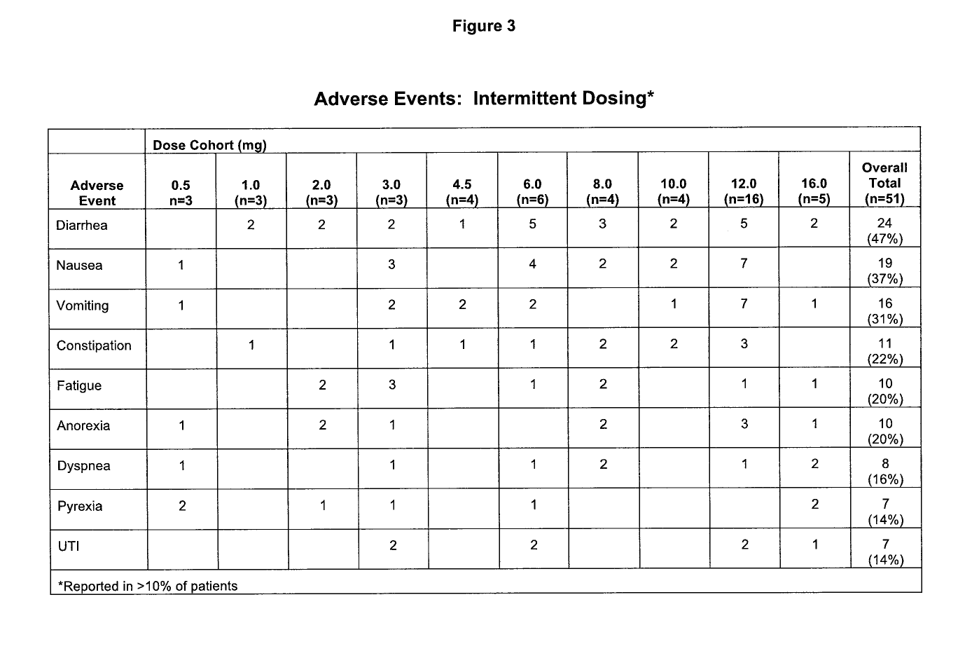

Most common adverse events (AEs) included diarrhea, nausea, vomiting, and constipation. FIG. 3 describes adverse events with intermittent dosing. Related Grade 3 events included vomiting (n=3), diarrhea (n=3), liver enzyme elevation (n=2) dehydration (n=1), and worsened hypertension (n=1).

No significant increase in toxicity was observed in patients receiving >2 cycles with intermittent dosing schedule.

The Maximal Tolerated Dose (MTD) for intermittent dosing was determined as 12 mg per day.

Safety Levels with Continuous Dosing

Continuous dosing schedule: The starting dose was two dose levels below the MTD of intermittent dosing (8 mg) and subsequent dose levels was one or two dose levels (10 mg or 8 mg) below the MTD of intermittent dosing dependent on recommendation of the dose cohort review committee. FIG. 1 illustrates the dosing schedule for continuous dosing.

At a dose of 10 mg per day, DLT was observed in 2/3 patients treated at 10 mg. Grade 3 diarrhea (n=2) was observed.

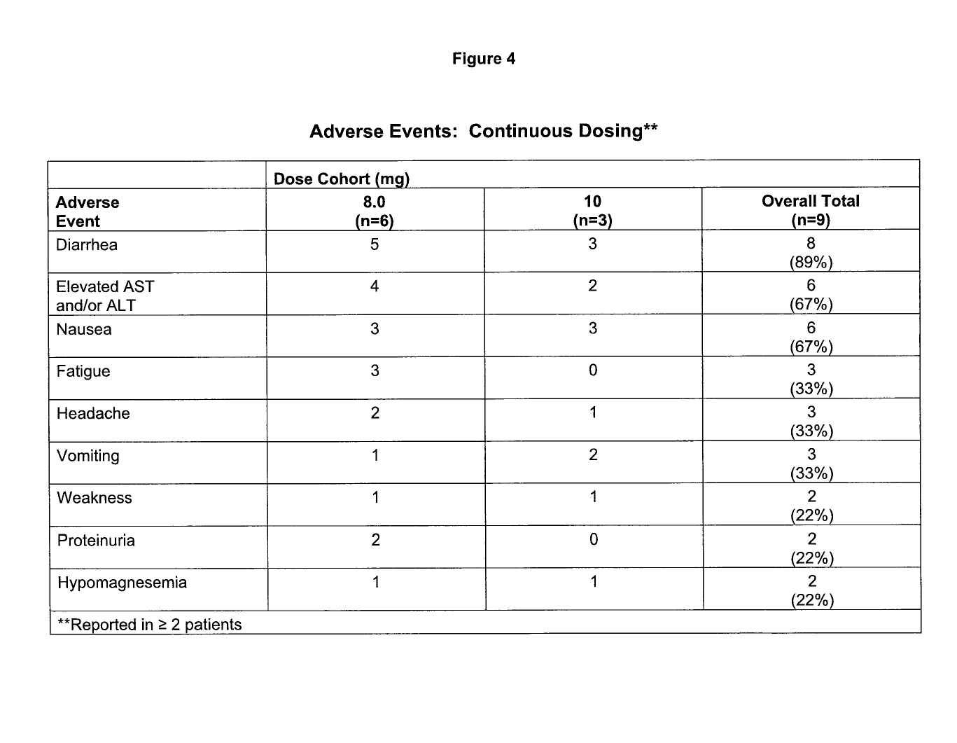

Most common adverse events (AEs) include diarrhea, nausea, vomiting, headache and fatigue. ALT/AST elevation was observed with continuous dosing. Related Grade 3 events include vomiting (n=3), diarrhea (n=3), liver enzyme elevation (n=2) dehydration (n=1), worsened hypertension (n=1). FIG. 4 describes adverse events with continuous dosing.

No significant increase in toxicity was observed in patients receiving >2 cycles with intermittent dosing schedule.

The Maximal Tolerated Dose (MTD) for continuous dosing was determined as 8 mg per day.

### Study Response

Patient response was evaluated according to Response Evaluation Criteria in Solid Tumors (RECIST). Briefly, all measurable lesions up to a maximum of five lesions, representative of all involved organs were identified as target lesions and recorded and measured at baseline. Target lesions were selected on the basis of their size (lesions with the longest diameter) and their suitability for accurate repeated measurements (either by imaging techniques or clinically). A sum of the longest diameter (LD) for all target lesions was calculated and reported as the baseline sum LD. The baseline sum LD was used as reference by which to characterize the objective tumor. All other lesions (or sites of disease) were identified as non-target lesions and were also be recorded at baseline. Measurements of these lesions were not required, but the presence or absence of each was noted throughout follow-up.

A Complete Response (CR) indicated a disappearance of all target lesions. A Partial Response (PR) showed at least a 30% decrease in the sum of the LD of target lesions, taking as reference the baseline sum LD. Progressive Disease (PD) was defined as at least a 20% increase in the sum of the LD of target lesions, taking as reference the smallest sum LD recorded since the treatment started or the appearance of one or more new lesions and Stable Disease (SD) indicated that there was neither sufficient shrinkage to qualify for PR nor sufficient increase to qualify for PD, taking as reference the smallest sum LD since the treatment started.

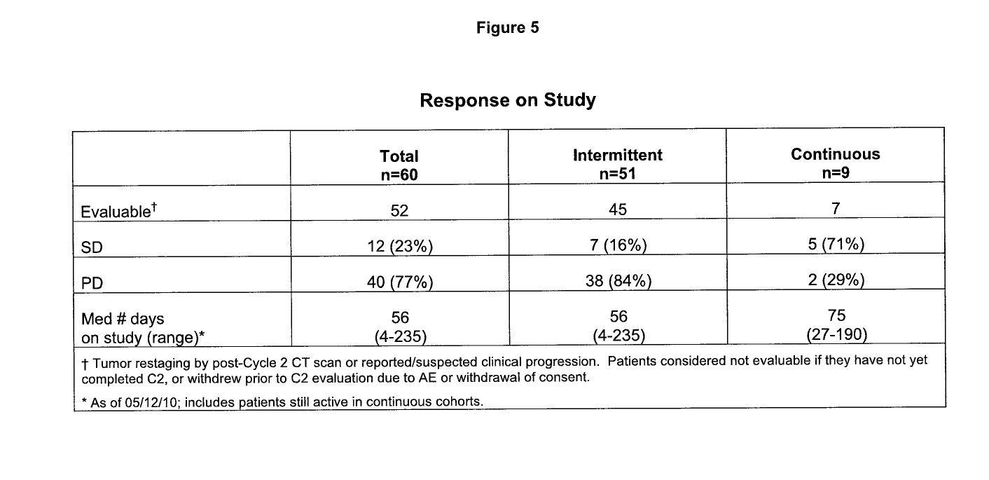

FIG. 5 describes response to intermittent and continuous dosing studies. Disease stabilization was observed in 71% of patients undergoing continuous dosing. Disease stabilization was observed in 16% of patients undergoing intermittent dosing.

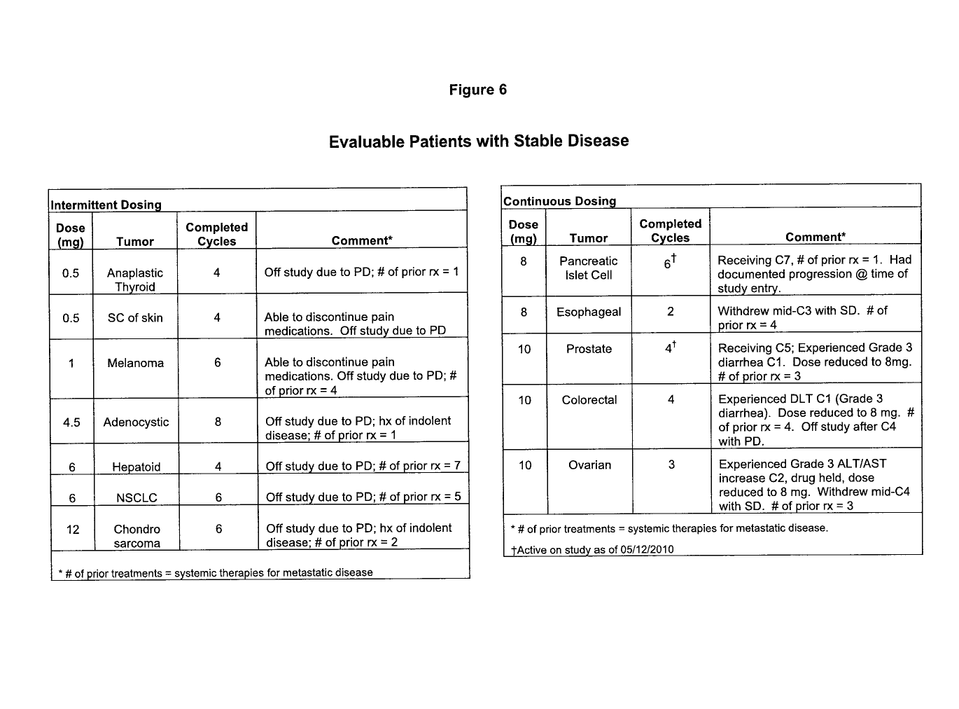

Patients, who were prior treated with other drugs but had subsequent disease progression, benefitted from treatment with PX-866. PX-866 treatment stabilized disease in prior treated patients. FIG. 6 describes evaluable patients with stable disease that had prior treatments. The number of prior treatments with other drugs ranged from 1 to 7 for these patients, with a median of 4 prior systemic therapeutic regimens for metastatic disease.

### Clinical Pharmacokinetics

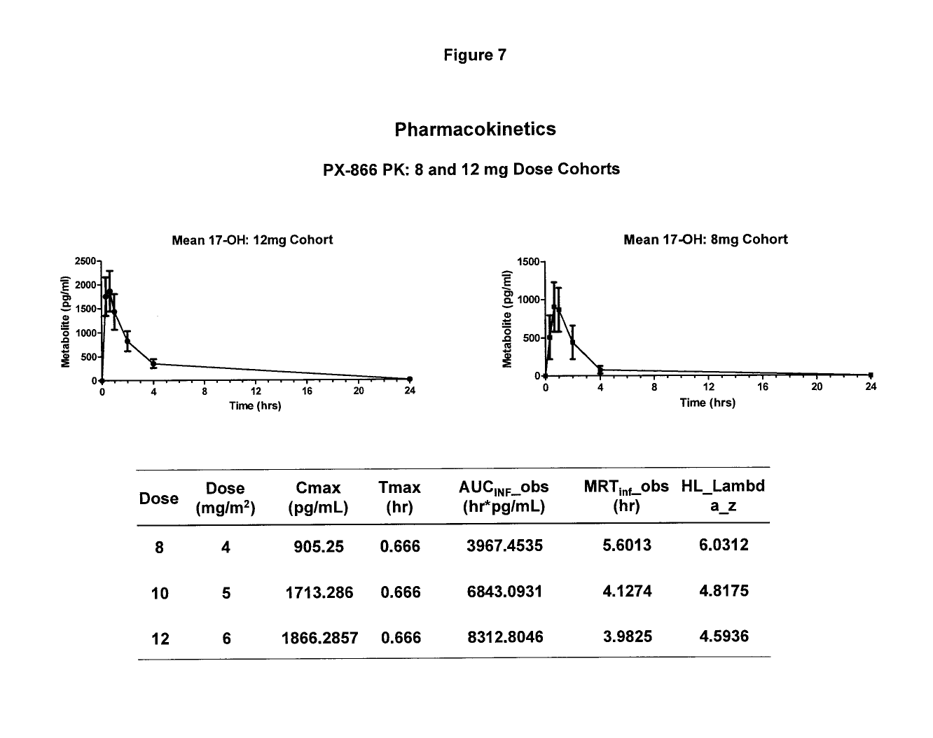

Pharmacokinetic studies revealed evidence of rapid conversion of PX-866 to a 17-OH metabolite. With rare exceptions, the parent PX-866 was below the limits of detection. Production of the 17-OH metabolite was rapid, with a Tₘₐₓ ranging from 0.67-1.07 hours. FIG. 7 depicts the pharmacokinetics of PX-866 administration in humans in 8 and 12 mg cohorts for intermittent dosing. Analysis of the 12 mg cohort revealed that the pharmacokinetics of the 17-OH metabolite showed no evidence of drug accumulation.

Analysis of data from patients dosed at the continuous dosing of 8 mg showed a mean Cₘₐₓ of 1140 pg/mL, an AUC₍₀₋₂₄₎ of 4220 hr*pg/mL and a half-life of 3.62 hr.

It was particularly noted that the Cmax of the 17-OH metabolite was equal to or exceed peak levels observed in mice treated at an efficacious dose of PX-866 (2 mg/kg). In addition the AUC for the 17-OH metabolite in humans exceeded AUC in mice due to an increase in mean residence time in humans.

### Clinical Pharmacodynamics

The pharmacodynamic effects of PX-866 in patients treated in the phase I single agent study were assessed using isolated peripheral blood mononuclear cells (PBMCs) stimulated ex vivo via FACS based assay. PX-866 treatment was associated with inhibition of the PI-3K pathway as assessed by changes in the downstream kinases p-mTOR and p-S6. The study provided Evidence for pathway inhibition lasting up to 3 days post-treatment.

Additional pharmacodynamic data from patients on the phase I study of PX-866 indicate that 3 of 4 patients treated at the 8 mg dose level of PX-866 had a 60% or greater decrease in p-AKT/T-AKT 4 hours after a single oral dose of drug.

### Example 2

### Effect of PX-866 in Subcutaneous and Intracranial Glioblastoma Xenograft Animal Model

PX-866 is examined in glioblastoma xenograft animal models to evaluate the effects of mean tumor volume and growth in subcutaneous U87 animal models and survival in intracranial U87 animal models. U87 glioblastoma xenografts are implanted subcutaneously (s.c.) or intracranially (i.c.) in nude mice similar to procedures previously described in Phuong et al, Canc Research, 2003 63: 2462-69. Briefly, in the subcutaneous U87 tumor model, about 3-5×10⁶ U87 cells are injected subcutaneously into the flanks of 4-week old nude mice. The mice are examined for tumor growth and size by calipers. For the intracranial U87 tumor model, injection of U87 cells into the caudate nucleus of nude mice is performed using a small animal stereotactic frame or guide screw system. s.c. and i.c. U87 animal models receive either a dose of PX-866 between 8-12 mg/kg IV and 2 to 4 mg/kg or vehicle alone.

### Example 3

### Phase 2 Study of PX-866 in Patients with Glioblastoma Multiforme at Time of First Relapse or Progression

### Study Objectives

1. To determine the efficacy of PX-866 given orally daily in patients with glioblastoma at the time of first relapse or progression as assessed by objective response and early progression rates.

2. To determine the safety and tolerability of PX-866 given in a daily oral schedule in patients with glioblastoma at first relapse/progression.

3. To explore the relationship between objective response and molecular markers in archival tissue from glioblastoma patients treated with PX-866 orally daily.

### Primary Endpoints

The primary endpoints of this study are objective response and progression as defined by MacDonald et al, J Clin Oncol. 1990; 8(7):1277-1280. Response is assessed by evaluation of change in product of bidimensional measurement of enhancing brain tumor on CT scan or MRI. A 50% decrease in the product is considered a partial response. Progression is a 25% increase in product.

### Study Population

Eligible patients are those with histologically confirmed diagnosis of glioblastoma multiforme (GBM), with recurrent or progressive disease following or during primary treatment not curable with standard therapies who meet all of the following inclusion criteria:

Inclusion Criteria:
≧18 years at time of consent
Able to give an informed consent
Fixed paraffin embedded tissue available for translational studies
Bidimensionally measurable enhancing lesions on CT or MRI, with at least one lesion with a minimum dimension of 1 cm×1 cm (i.e. both dimensions must be >1.0 cm)
Eastern Cooperative Oncology Group (ECOG) performance of 0, 1 or 2
Prior therapy:
   Chemotherapy: May have received prior adjuvant chemotherapy and/or concurrent chemoradiation as part of primary therapy, but must have received no therapy for recurrent/progressive GBM (i.e. PX-866 must be first treatment for recurrence/progression). A minimum of 28 days since the last dose of chemotherapy must have elapsed prior to registration.
   Targeted therapy: No prior therapy with a phosphatidylinositol 3-kinase (PI-3K) inhibitor. Other targeted agents are permissible provided they were given as part of front line treatment. A minimum of 56 days (8 weeks) must have elapsed since last day for anti-angiogenic therapy and minimum of 28 days for other targeted agents
   Radiation: Patients may have had prior radiation therapy provided at least 28 days have elapsed from the day of the last fraction of radiation to the date of registration.
   Previous surgery: Previous surgery is permitted provided that wound healing has occurred and at least 14 days have elapsed prior to registration.
Laboratory requirements:

| | |
|---|---|
| Granulocytes (AGC) | ≧1.5 × 10⁹/L |
| Platelets | ≧100 × 10⁹/L |
| Serum creatinine | ≦1.5 × UNL |
| Total bilirubin | ≦1.5 × UNL |
| Aminotransferases (ALT and AST) | ≦1.5 × UNL |
| Glucose | ≦8.9 mmol/L (≦Grade 1) |

Women must be post menopausal, surgically sterile or use a reliable form of contraception while on study and for 30 days after discontinuing therapy. Women of childbearing potential must have a pregnancy test taken and proven negative within 7 days prior to registration and must not be lactating.

Exclusion Criteria:
Patients who have other active malignancies (i.e. documented by imaging, clinical exam or marker) are to be excluded
Known human immunodeficiency virus (HIV) positive
Uncontrolled diabetes mellitus
Patients should be on a stable dose of steroid (i.e. no change in dose for 2 weeks prior to registration) when entered on study. Patients recently started on steroids or whose steroid dose was increased in the recent past should not be started on protocol treatment until at least 2 weeks have passed from the time of steroid dose increment or initiation.
Patients with upper gastrointestinal or other conditions that would preclude compliance or absorption of oral medication are not eligible.
Patients with active or uncontrolled infections, or with serious illnesses or medical conditions which would not permit the patient to be managed according to the protocol
Patients are not eligible if they have a known hypersensitivity to the study drugs or their components.
Previous treatment with a phosphatidylinositol 3-kinase (PI-3K) inhibitor

### Study Design

### Pre-Treatment Evaluations

Prior to treatment, a patient undergoes pre-treatment evaluations including history, physical exam, hematology and biochemistry, toxicity/baseline symptoms, urinalysis and pregnancy test (within 7 days prior to patient registration). A CT or MRI brain scan and a neurological examination are also taken.

### Treatment

After establishing eligibility, patients are enrolled in the current dose cohort of 8 mg PX-866 administered orally in capsule form on a daily schedule. 1 reporting period=1 cycle=8 weeks. Patients swallow the capsules whole with approximately 250 ml of water every day (preferably at the same time each day). In this study, PX-866 is administered with water to patients on an empty stomach (≧1 hour before a meal or >2 hours after).

On treatment evaluations include hematology and biochemistry (Cycle 1: weekly for 4 weeks, thereafter every 2 weeks; Cycle 2: every 2 weeks; Cycle 3+: every 4 weeks), neurological exam (end of every cycle), urinalysis (Day 1 of each cycle), physical exam (weight, blood pressure, heart rate, pulse, ECOG performance; Cycle 1: Days weekly for 5 weeks; Cycle 2+: every 4 weeks) tumor assessment (CT or MRI brain scan every 8 weeks) and toxicity assessment (every visit).

### Treatment Duration

For complete responders, therapy continues until progression or for 8 weeks after CR criteria are first met. For partial responders, therapy continues until progression or for 8 weeks after documentation of stable partial response (i.e. no further tumor shrinkage documented). For stable patients, therapy continues for a maximum of 48 weeks (6 cycles). Patients who have no evidence of response at this point are recommended to go off therapy and receive other treatment at the investigator's discretion. Patients who progress (treatment failure) will go off study at the time progression is documented clinically and/or radiographically.

### Response Definition

Once CT/MRI scan and clinical assessment is complete, patients are classified and managed according to the following table:

| | CLINICAL NEUROLOGIC ASSESSMENT | | |
|---|---|---|---|
| | | Equivocal/No | |
| CT or MRI SCAN | Better | Change | Worse |
| Disappearance of | Complete response | Complete response | investigate |
| enhancing lesion | (continue therapy) | (continue therapy) | |
| and mass effect | | | |
| Definite | Partial response | Partial response | investigate |
| improvement | (continue therapy) | (continue therapy) | |
| (≧50% decrease) | | | |
| Equivocal/no change | Stable | Stable | investigate |
| (<50% decrease | (continue therapy) | (continue therapy) | |
| and <25% increase) | | | |
| Definite progression | investigate | Progression | Progression |
| (≧25% increase) | | (off protocol therapy) | (off protocol therapy) |

### Dose Adjustments

Doses are reduced for hematologic and other adverse events. Dose adjustments are made according to the system showing the greatest degree of toxicity. Adverse events are graded using the NCI Common Terminology Criteria for Adverse Events (CTCAE) Version 4.0. A dose reduction schedule is provided below.

| Dose Level | Daily Dose |
|---|---|
| 0 | 8 mg |
| −1 | 6 mg |
| −2 | 4 mg |

The following table illustrates dosage adjustment criteria for this study:

Hematological Adverse Events

| Absolute | | | |
|---|---|---|---|
| Granulocytes | | Platelets | |
| (×10⁹/L) | | (×10⁹/L) | PX- 866 Treatment |
| <1.0 | OR | <50 | Hold dose until recovery to >1.5 granulocytes and >75 platelets. |
| | | | If continued therapy is planned, then reduce * one dose level. |

* If no recovery after a 2 week delay, patient should go off protocol treatment.
Patients requiring more than 2 dose reductions should go off protocol treatment.

Elevation in ALT or AST

| | |
|---|---|
| Grade 3 ALT and/or AST | Hold* until severity ≦ Grade 1 |
| OR | If continued therapy is planned, then reduce** one dose level. |
| Grade 2 ALT and/or AST | |
| AND >Grade 2 bilirubin*** | |
| OR | |
| Grade 3 bilirubin | |
| Grade 3 ALT and/or AST | Off protocol therapy |
| AND >Grade 2 bilirubin* | |
| OR | |
| Grade 4 ALT and/or AST | |
| OR | |
| Grade 4 bilirubin | |

*If no recovery after a 2 week delay, patient should go off protocol treatment.
**Patients requiring more than 2 dose reductions should go off protocol treatment.
***Elevated bilirubin must be due to treatment and not Gilbert’s disease

Nausea/Vomiting

| | |
|---|---|
| Grade 3 nausea, vomiting or | Hold* dose until recovery to Grade 1 or baseline grade |
| diarrhea WITHOUT maximal use | Initiate appropriate supportive therapy |
| of anti-emetics or anti-diarrheals | If continued therapy is planned, restart treatment at same dose |
| | level but with supportive therapy. If grade 3 nausea, vomiting |
| | or diarrhea recurs, follow same algorithm but reduce** dose by |
| | one dose level following recovery |
| Grade 3 nausea, vomiting or | Hold dose until recovery to ≦Grade 1 |
| diarrhea WITH maximal use of | If continued therapy is planned, then reduce** one dose level |
| anti-emetics or anti-diarrheals | |
| Grade 4 | Off protocol therapy |

*If no recovery after a 2 week delay, patient should go off protocol treatment.
**Patients requiring more than 2 dose reductions should go off protocol treatment.

Other Non-hematological Adverse Events

| | |
|---|---|
| Grade 3 AEs other than alopecia, | Hold* dose until recovery to ≦Grade 1 or baseline |
| nausea, vomiting or diarrhea. | If continued therapy is planned, then reduce** one dose level |
| Grade 4 | Off protocol therapy |

*If no recovery after a 2 week delay, patient should go off protocol treatment.
**Patients requiring more than 2 dose reductions should go off protocol treatment.

Dose reductions or treatment interruption for reasons other than those described above are made by the clinical investigator if it is deemed in the best interest of patient safety. Whenever possible, these decisions are first discussed with the study medical monitor.

Doses held for toxicity are not replaced. Doses reduced for toxicity are not re-escalated. In general when treatment is withheld because of drug related adverse effects for >2 weeks without recovery to the degree required for restarting treatment, the patient should go off protocol therapy.

### Statistical Methods

This study accrues up to 30 patients. A multinomial stopping rule incorporating both response and early progression are employed in a 2-stage design. In the first stage, 15 evaluable patients are enrolled (includes patients enrolled at recommended dose of phase I part of trial). If there are 0 responses AND 10 or more early progressions, entry is stopped. If there are 1 or more responses OR<10 early progressions, that arm is continued and 15 more patients are entered (second stage).

Significance Level and Power:

The procedure described above tests the null hypothesis that the response rate is ≦5% and early progression rate ≧60% versus alternative hypotheses that the response rate is ≧20% and early progression rate is ≦30%. If the true response rate is 5% and the true progression rate is 60%, the level of significance of the above rule, i.e. the probability of concluding the drug is interesting when it is not active, is 0.1; and if the true response rate is 20% and the true progression rate is 40% the power of the above rule, i.e. the probability of concluding the drug is interesting when it is active, is 0.93.

Correlative Studies and Identification of Biomarkers:

Archival tissue is assayed for PTEN, EGFRvIII, PIK3CA mutations and other potential markers of PI-3K inhibitory effect using immunohisto-chemistry (IHC) and/or FISH and/or mutational analysis. Chi-square (categorical results) or logistic regression models (continuous results) will be used to explore the relationship between archival findings with tumor response or early progression.

### Example 4

### Effect of PX-866 on the Rate of Cell Proliferation of Androgen-Independent LnCaP Cells In Vitro

PX-866 is investigated for the effects on cell proliferation rates of an androgen independent prostate cancer cell line, LnCaP C4-2B. C4-2B cells are plated in 96-well plates at a density of 300,000 cells per well in RPMI medium containing 5% CSS for 1 day. On the following day, the cells are treated with PBS vehicle, PX-866, wortmannin and a previously reported cell proliferation inhibitor, cyclopamine as a positive control. For the drugs, the cells are exposed at various concentrations that range from about 10⁻⁷-10⁻³M for 72 hours to determine IC₅₀ concentrations. The IC₅₀ is defined as the concentration of drug at which there is a 50% less growth when compared to control cells. Each experiment is performed in triplicate.

After 72 hours drug exposure, the cell media with drug is removed and cell proliferation is determined via MTT [3-(4,5-dimethylthiazol-2-yl)-2,5-diphenyltetrazolium bromide] assay. The MTT assay is based on the ability of a mitochondrial dehydrogenase enzyme from viable cells to cleave the tetrazolium rings of the pale yellow MTT and form dark blue formazan crystals which are largely impermeable to cell membranes, thus resulting in its accumulation within viable cells. The color can then be quantified using a colorimetric assay. Briefly, 20 μl of 5 mg/ml MTT substrate is added to each well. Plates are returned to the incubator and left in the dark for 1 hour. After the incubation period, MTT substrate/medium is gently removed from each well and 200 μl of DMSO is added to each well to dissolve the MTT formazan crystals and absorbance measured spectrophotometrically at a wavelength of 570 nm. Blank control values are then subtracted from the 570 nm values and relative growth rates were calculated.

### Example 5

### Effect of PX-866 on a Castration Resistant Prostate Tumor Xenograft Animal Model

PX-866 is examined in castration resistant prostate tumor xenograft animal models to evaluate the effects of mean tumor volume and growth and changes in prostate specific antigen (“PSA”) levels. Castration resistant prostate tumor xenografts are made by (3×10⁶ LnCaP C₄₋₂B cells) are implanted subcutaneously (s.c.) in 6 to 8 week old male athymic nude mice (Harlan Sprague Dawley, Inc.) via a 27-gauge needle under halothane anesthesia. Tumor volume and serum PSA measurements (blood collected from the tail vein) were performed once per week after tumours became palpable. PSA levels were measured by ELISA (ClinPro International) and tumor size by calipers. Once serum PSA values reached 75-100 ng/mL, mice receive either a dose between 8-12 mg/kg IV or 2 to 4 mg/kg orally daily of PX-866 or vehicle alone. Each animal group contains a minimum of 4 mice, with a range of 4-6 mice. PSA measurements are used to calculate PSA velocity and volume measurements are used to determine the tumor growth rate for all groups with linear regression slope analysis. PSA velocity is defined as the increase in PSA level (normalized to pre-treatment value set at 100%) divided by number of days that PSA is reliably measurable. Tumor growth rate is defined as the increase in tumor volume (normalized to pre-treatment value set at 100%) divided by the duration of the experiment.

### Example 6

### Phase 2 Study of PX-866 in Patients with Castration Resistant Prostate Cancer

### Study Objectives

1. To determine the efficacy of PX-866 given orally daily in patients with castration resistant prostate cancer who have received no prior chemotherapy regimens for recurrent disease.

2. To determine the safety and tolerability of PX-866 given in a daily oral schedule in patients with castration resistant prostate cancer.

3. To explore the relationship between objective response and molecular markers in archival tissue from castration resistant prostate cancer patients treated with PX-866 orally daily.

4. To investigate additional potential measures of efficacy including PSA response rate, objective response rate (in patients with measurable disease at baseline, and change in circulating tumor cell number during treatment.

### Primary Endpoints

The primary endpoints of this study are is the assessment of efficacy as measured by a PSA decline of ≧50% or lack of disease progression at 12 weeks. A multinomial design utilizing response and early progression is employed for this study.

### Study Population

Eligible patients are those with histological or cytological diagnosis of adenocarcinoma of the prostate who meet the following inclusion/exclusion criteria:

Inclusion Criteria:
≧18 years at time of consent
Able to give an informed consent
Radiologic and/or clinically documented evidence of metastatic disease.
Formalin fixed paraffin embedded tissue from primary or metastatic tumor for translational studies
Have metastatic or locally recurrent disease for which no curative therapy exists and for which systemic therapy is indicated due to progression (2 definitions described below) following castration
   PSA progression:
   A rising PSA, while receiving androgen ablative therapy, with two consecutive rises (PSA-1, PSA-2) from a baseline measurement (PSA-b) measured at least 1 week apart where PSA-b<PSA-1<PSA-2. If PSA-2>PSA-b but PSA-2 is <PSA-1, a third PSA rise (PSA-3) is also acceptable as evidence of progression provided PSA-3 is >PSA-1 and PSA-2. The last PSA documenting progression (PSA 2 or 3) must be performed within 7 days of registration.
   OR
   Radiological progression: development of new metastatic lesions with a stable or rising PSA
Castration therapy (androgen ablation) must include either medical or surgical castration. If the patient is receiving medical androgen ablation, a castrate level of testosterone (≦1.7 nmol/L) must be present.
PSA ≧5 ng/mL at the time of study entry
Eastern Cooperative Oncology Group (ECOG) performance of 0, 1 or 2
Prior therapy:
   Surgery: Patients must be >2 weeks since any major surgery.
   Chemotherapy: No prior cytotoxic chemotherapy is permitted for recurrent/metastatic castration resistant prostate cancer. Prior hormone therapy is required. Patients must have discontinued anti-androgens for at least 4 weeks prior to study entry (at least 6 weeks for bicalutamide). Prior therapy with CYP17 inhibitors (e.g. abiraterone, ketoconazole) or novel anti-androgens (e.g. MDV3100) is permitted.
   Radiation: Prior external beam radiation is permitted provided a minimum of 2 weeks has elapsed between the last dose and enrolment to the trial.
Laboratory requirements:

| | |
|---|---|
| Granulocytes (AGC) | ≧1.5 × 10⁹/L |
| Platelets | ≧100 × 10⁹/L |
| Serum creatinine | ≦1.5 × UNL |
| Total Bilirubin | ≦1.5 × UNL |
| Aminotransferases (ALT and AST) | ≦1.5 × UNL |
| Glucose | ≦8.9 mmol/L (≦Grade 1) |

Exclusion Criteria:
History of other malignancies, except: adequately treated non-melanoma skin cancer or solid tumours curatively treated with no evidence of disease for ≧3 years.
HIV-positive
Uncontrolled diabetes mellitus
Upper gastrointenstinal or other conditions that would preclude compliance or absorption of oral medication
Active or uncontrolled infections or with serious illnesses or medical conditions which would not permit the patient to be managed
Known hypersensitivity to the study drug(s) or the their components
History of CNS metastases or untreated spinal cord compression
Prior treatment with a P-13 kinase inhibitor
Not sterile unless an adequate method of birth control is used

### Study Design

### Pre-Treatment Evaluations

Prior to treatment, a patient undergoes pre-treatment evaluations including history, physical exam, hematology and biochemistry, toxicity/baseline symptoms, urinalysis and PSA measurement (within 7 days prior to patient registration). A chest/pelvic CT or MRI scan and a bone scan is also taken. Other scans/x-rays as necessary are taken to document disease.

### Treatment

After establishing eligibility, patients are enrolled in the current dose cohort of 8 mg PX-866 administered orally in capsule form on a daily schedule. 1 reporting period=1 cycle=6 weeks. Patients swallow the capsules whole with approximately 250 ml of water every day (preferably at the same time each day). In this study, PX-866 is administered with water to patients on an empty stomach (>1 hour before a meal or >2 hours after).

On treatment evaluations include hematology (Day 1 of each cycle) and biochemistry (Day 1 and Day 15 of each cycle for 2 cycles then Day 1 each cycle), PSA measurement (every 4 weeks), urinalysis (Day 1 of each cycle), physical exam (weight, blood pressure, heart rate, pulse, ECOG performance; Cycle 1: weekly; Cycle 2+: every 2 weeks); Bone scan (baseline and every 12 weeks), tumor assessment (pelvic CT or MRI scan every 12 weeks) and toxicity assessment (every visit).

### Treatment Duration

Patients receive treatment until tumor progression or unacceptable toxicity. In absence of toxicity or disease progression, patients continue on therapy for a maximum of 6 reporting periods.

### Response Definition

Objective response and outcome measures as described in Scher et al, J Clin Oncol 26:1148-1159, 2008. Response is assessed by response according to RECIST and/or PSA response.

RECIST Response Definition: Complete Response (CR) is defined as the disappearance of target and non-target lesions and normalization of tumor markets. Partial Response (PR) is at least a 30% decrease in the sum of measures (longest diameter for tumor lesions and short axis measure for nodes) of target lesions, with respect to baseline sum of diameters. Stable Disease (SD) is defined as neither sufficient shrinkage to qualify for PR nor sufficient increase to qualify for Progressive Disease. Progressive Disease (PD) is at least a 20% increase in the sum of diameters of measured lesions with reference to the smallest sum of diameters recorded on study (including baseline) AND an absolute increase of ≧5 mm.

PSA Response Criteria: PSA Response is defined as PSA decline from baseline of 50% decrease maintained for ≧4 weeks. PSA Progression is 25% increase PSA from baseline/nadir and is confirmed by a second increasing value at least 3 weeks later. Non-response is failure to achieve PSA response criteria.

Once response or progression is assessed, patients can be classified and managed according to the following table:

| | CLINICAL ASSESSMENT | | |
|---|---|---|---|
| PSA Measurement | | Equivocal/No | |
| OR RECIST | Better | Change | Worse |
| PSA ≧50% decrease | Complete response | Complete response | investigate |
| OR RECIST | (continue therapy) | (continue therapy) | |
| Definite | Partial response | Partial response | investigate |
| improvement | (continue therapy) | (continue therapy) | |
| PSA ≧25% decrease | | | |
| Equivocal/no change | Stable | Stable | investigate |
| PSA <25% decrease | (continue therapy) | (continue therapy) | |
| and <25% increase | | | |
| Definite progression | investigate | Progression | Progression |
| PSA ≧25% increase | | (off protocol therapy) | (off protocol therapy) |

### Dose Adjustments

Doses are reduced for hematologic and other adverse events. Dose adjustments are made according to the system showing the greatest degree of toxicity. Adverse events are graded using the NCI Common Terminology Criteria for Adverse Events (CTCAE) Version 4.0. A dose reduction schedule is provided below.

| Dose Level | Daily Dose |
|---|---|
| 0 | 8 mg |
| −1 | 6 mg |
| −2 | 4 mg |

The following table illustrates dosage adjustment criteria for this study:

Hematological Adverse Events

| Absolute | | | |
|---|---|---|---|
| Granulocytes | | Platelets | |
| (×10⁹/L) | | (×10⁹/L) | PX- 866 Treatment |
| <1.0 | OR | <50 | Hold dose until recovery to >1.5 granulocytes and >75 platelets. |
| | | | If continued therapy is planned, then reduce * one dose level. |

* If no recovery after a 2 week delay, patient should go off protocol treatment.
Patients requiring more than 2 dose reductions should go off protocol treatment.

Elevation in ALT or AST

| | |
|---|---|
| Grade 3 ALT and/or AST | Hold* until severity ≦ Grade 1 |
| OR | If continued therapy is planned, then reduce** one dose level. |
| Grade 2 ALT and/or AST | |
| AND >Grade 2 bilirubin*** | |
| OR | |
| Grade 3 bilirubin | |
| Grade 3 ALT and/or AST | Off protocol therapy |
| AND >Grade 2 bilirubin* | |
| OR | |
| Grade 4 ALT and/or AST | |
| OR | |
| Grade 4 bilirubin | |

*If no recovery after a 2 week delay, patient should go off protocol treatment.
**Patients requiring more than 2 dose reductions should go off protocol treatment.
***Elevated bilirubin must be due to treatment and not Gilbert's disease

Nausea/Vomiting

| | |
|---|---|
| Grade 3 nausea, vomiting or | Hold* dose until recovery to Grade 1 or baseline grade |
| diarrhea WITHOUT maximal use | Initiate appropriate supportive therapy |
| of anti-emetics or anti-diarrheals | If continued therapy is planned, restart treatment at same dose |
| | level but with supportive therapy. If grade 3 nausea, vomiting |
| | or diarrhea recurs, follow same algorithm but reduce** dose by |
| | one dose level following recovery |
| Grade 3 nausea, vomiting or | Hold dose until recovery to ≦Grade 1 |
| diarrhea WITH maximal use of | If continued therapy is planned, then reduce** one dose level |
| anti-emetics or anti-diarrheals | |
| Grade 4 | Off protocol therapy |

*If no recovery after a 2 week delay, patient should go off protocol treatment.
**Patients requiring more than 2 dose reductions should go off protocol treatment.

Other Non-hematological Adverse Events

| | |
|---|---|
| Grade 3 AEs other than alopecia, | Hold* dose until recovery to ≦Grade 1 or baseline |
| nausea, vomiting or diarrhea. | If continued therapy is planned, then reduce** one dose level |
| Grade 4 | Off protocol therapy |

*If no recovery after a 2 week delay, patient should go off protocol treatment.
**Patients requiring more than 2 dose reductions should go off protocol treatment.

Dose reductions or treatment interruption for reasons other than those described above are made by the clinical investigator if it is deemed in the best interest of patient safety. Whenever possible, these decisions are first discussed with the study medical monitor.

Doses held for toxicity are not replaced. Doses reduced for toxicity are not re-escalated. In general when treatment is held because of drug related adverse effects for >2 weeks without recovery to the degree required for restarting treatment, the patient should go off protocol therapy.

### Statistical Methods

This study accrues up to 40 patients. A multinomial stopping rule incorporating both response and early progression are employed in a 2-stage design. In the first stage, 15 evaluable patients are enrolled (includes patients enrolled at recommended dose of phase I part of trial). If there are 0 responses AND 10 or more early progressions, entry is stopped. If there are 1 or more responses OR<10 early progressions, that arm is continued and 15 more patients are entered (second stage).

Significance Level and Power:

The procedure described above tests the null hypothesis that the response rate is ≦5% and early progression rate ≧60% versus alternative hypotheses that the response rate is ≧20% and early progression rate is ≦30%. If the true response rate is 5% and the true progression rate is 60%, the level of significance of the above rule, i.e. the probability of concluding the drug is interesting when it is not active, is 0.1; and if the true response rate is 20% and the true progression rate is 40% the power of the above rule, i.e. the probability of concluding the drug is interesting when it is active, is 0.93.

Correlative Studies and Identification of Biomarkers:

All patients enrolled to the study will have representative sections from their paraffin block of their primary diagnostic tumour specimen sent for evaluation. Archival tissue is assayed for PTEN, EGFRvIII, PIK3CA mutations and other potential markers of PI-3K inhibitory effect using immunohisto-chemistry (IHC) and/or FISH and/or mutational analysis. Copy number of the androgen receptor is also examined. Chi-square (categorical results) or logistic regression models (continuous results) will be used to explore the relationship between archival findings with tumor response or early progression.

Whole blood is collected at baseline (prior to cycle 1, day 1 dosing), at 6 weeks, and again at 12 weeks (for patients still in treatment). Circulating tumor cells (“CTC”) are evaluated for PTEN status and compared with results from that same patient in archival tissues. The relationship between CTC number and baseline patient factors, PSA changes, radiological response (for patients with measurable disease) and clinical progression is explored as well as the relationship between PTEN status of CTC and/or archival tissue and baseline patient factors, PSA changes, radiological response (for patients with measurable disease) and clinical progression.

The ratio of phosphorylated AKT versus total AKT in human platelets is also used as one pharmacodynamic measure of activity of PX-866 in these patients.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for treatment of cancer comprising administering to a human in need thereof a PX-866 compound of the following formula: 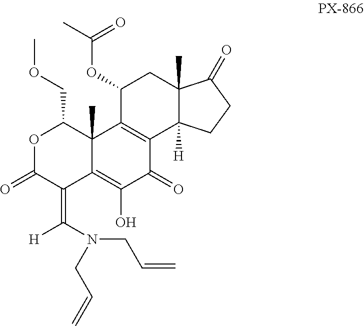 [SMILES: C=CCN(/C=C1/C(=O)O[C@H](COC)[C@@]2(C)C1=C(O)C(=O)C1=C2[C@H](OC(C)=O)C[C@]2(C)C(=O)CC[C@@H]12)CC=C]
at a dose and frequency of administration sufficient to result in a plasma concentration of a 17-hydroxy metabolite between about 500 pg/mL and about 2500 pg/mL (peak) within about 1-3 hours of administration of PX-866.

2. The method of claim 1, wherein PX-866 is administered to the human in an amount of from about 0.1 mg to about 20 mg per day.

3. The method of claim 1, wherein PX-866 is administered as a continuous dose, an intermittent dose or a combination thereof.

4. The method of claim 1, wherein a continuous dose is between about 10% and about 85% of the Maximum Tolerated Dose (MTD) of the intermittent dose.

5. The method of claim 1, wherein administration of PX-866 provides a plasma Cₘₐₓ of the 17-hydroxy metabolite of between about 750 pg/mL and about 1750 pg/mL.

6. The method of claim 1, wherein administration of PX-866 provides an AUC of between about 2000 hr*pg/mL and about 8000 hr*pg/mL for the 17-hydroxy metabolite.

7. The method of claim 1, wherein the cancer is selected from anaplastic thyroid tumor, sacrcoma of the skin, melanoma, adenocystic tumor, hepatoid tumor, non-small cell lung cancer, chondrosarcoma, pancreatic islet cell tumor, esophageal cancer, prostate cancer, ovarian cancer, squamous cell carcinoma of the head and neck, colorectal carcinoma, glioblastoma, cervical carcinoma, endometrial carcinoma, gastric carcinoma, and breast carcinoma.

8. The method of claim 1, wherein PX-866 is administered as a continuous dose of between about 2 mg to about 12 mg per day.

9. The method of claim 1, wherein the cancer is glioblastoma.

10. The method of claim 1, wherein the cancer is prostate cancer and wherein the prostate cancer is castration resistant.

11. A method for treating a human subject with a glioblastoma comprising administering to the subject a therapeutically effective compound selected from 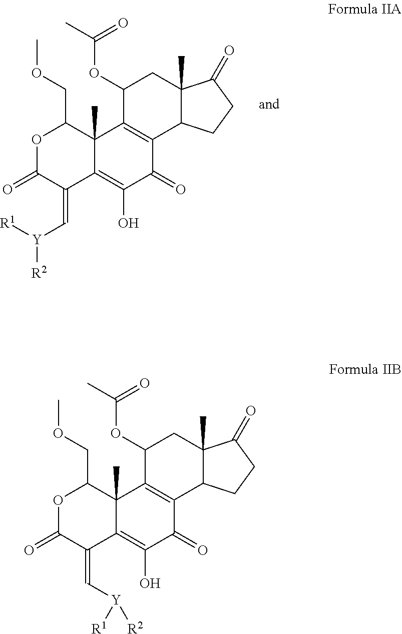
wherein Y is a heteroatom selected from nitrogen and sulfur and R¹ and R² are independently selected from an unsaturated alkyl, cyclic alkyl, or R¹ and R² together with Y form a_heterocycle.

12. The method of claim 11, wherein the glioblastoma is recurrent, metastatic, or unresectable.

13. The method of claim 11, wherein the administering is over a period of time selected from the group consisting of at least about 3 weeks, at least about 6 weeks, at least about 8 weeks, at least about 12 weeks, at least about 16 weeks, at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 68 weeks, at least about 72 weeks, at least about 90 weeks, at least about 100 weeks, at least about 110 weeks, and at least about 120 weeks.

14. The method of claim 11 further comprising evaluating the treated subject, wherein the evaluation comprises determining at least one of: (a) glioblastoma size, (b) glioblastoma location, (c) nodal stage, (d) growth rate of the glioblastoma, (e) survival rate of the subject, (f) changes in the subject's glioblastoma symptoms, (g) changes in the subject's biomarkers, or (h) changes in the subject's quality of life.

15. The method of claim 11, wherein the compound is

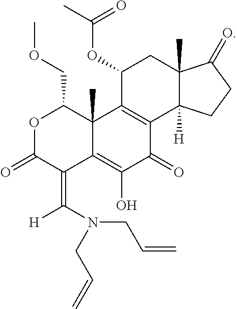 [SMILES: C=CCN(/C=C1/C(=O)O[C@H](COC)[C@@]2(C)C1=C(O)C(=O)C1=C2[C@H](OC(C)=O)C[C@]2(C)C(=O)CC[C@@H]12)CC=C]

16. A method for treating a human subject with a castration resistant prostate cancer comprising administering to the subject a therapeutically effective compound selected from 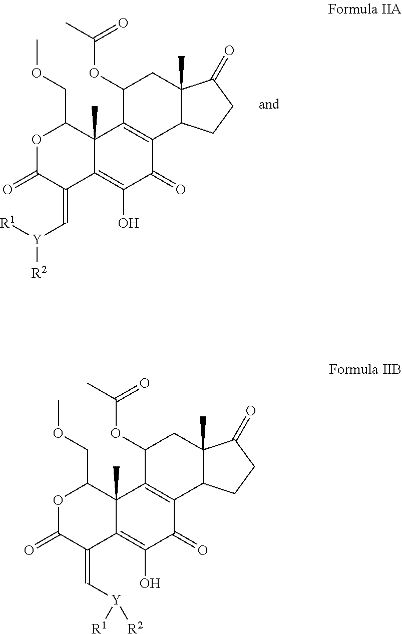
wherein Y is a heteroatom selected from nitrogen and sulfur and R¹ and R² are independently selected from an unsaturated alkyl, cyclic alkyl, or R¹ and R² together with Y form a heterocycle.

17. The method of claim 16, wherein the castration resistant prostate cancer is recurrent, metastatic, or unresectable.

18. The method of any one of claim 16, wherein the administering is over a period of time selected from the group consisting of at least about 3 weeks, at least about 6 weeks, at least about 8 weeks, at least about 12 weeks, at least about 16 weeks, at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 68 weeks, at least about 72 weeks, at least about 90 weeks, at least about 100 weeks, at least about 110 weeks, and at least about 120 weeks.

19. The method of claim 16 further comprising evaluating the treated subject, wherein the evaluation comprises determining at least one of: (a) tumor size, (b) tumor location, (c) nodal stage, (d) growth rate of the cancer, (e) survival rate of the subject, (f) changes in the subject's cancer symptoms, (g) changes in the subject's Prostate Specific Antigen (PSA) concentration, (h) changes in the subject's PSA concentration doubling rate, (i) changes in the subject's biomarkers, or (i) changes in the subject's quality of life.

20. The method of claim 16, wherein the compound is 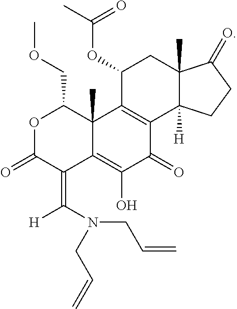 [SMILES: C=CCN(/C=C1/C(=O)O[C@H](COC)[C@@]2(C)C1=C(O)C(=O)C1=C2[C@H](OC(C)=O)C[C@]2(C)C(=O)CC[C@@H]12)CC=C]
